SEQUENCE LISTING (39 sequences)

SEQ ID NO: 1 - DNA, 33 bp, synthetic construct
  source 1..33: mol_type = other DNA
ttgtaggtagaagccatgggttttgatccactg

SEQ ID NO: 2 - DNA, 43 bp, synthetic construct
  source 1..43: mol_type = other DNA
caccttactttgccgtcacaagcttctacatttggtcccagtc

SEQ ID NO: 3 - DNA, 23 bp, synthetic construct
  source 1..23: mol_type = other DNA
aacagtggcttatgttcctcaag

SEQ ID NO: 4 - DNA, 23 bp, synthetic construct
  source 1..23: mol_type = other DNA
aactcctctttctccaatctctg

SEQ ID NO: 5 - DNA, 21 bp, synthetic construct
  source 1..21: mol_type = other DNA
agcccaagtaaagaggctaac

SEQ ID NO: 6 - DNA, 21 bp, synthetic construct
  source 1..21: mol_type = other DNA
tacaacagggctgcttagaac

SEQ ID NO: 7 - DNA, 20 bp, synthetic construct
  source 1..20: mol_type = other DNA
cagaacagcaggtcgaagac

SEQ ID NO: 8 - DNA, 19 bp, synthetic construct
  source 1..19: mol_type = other DNA
ccattgctcaaggccatct

SEQ ID NO: 9 - DNA, 19 bp, synthetic construct
  source 1..19: mol_type = other DNA
gcacgctcttcttgctttc

SEQ ID NO: 10 - DNA, 20 bp, synthetic construct
  source 1..20: mol_type = other DNA
agggaatcttgtcagggttg

SEQ ID NO: 11 - DNA, 21 bp, synthetic construct
  source 1..21: mol_type = other DNA
agcctgaactcaccgcgacgt

SEQ ID NO: 12 - DNA, 24 bp, synthetic construct
  source 1..24: mol_type = other DNA
tacttctacacagccatcggtcca

SEQ ID NO: 13 - DNA, 23 bp, synthetic construct
  source 1..23: mol_type = other DNA
cattgcggacgtttttaatgtac

SEQ ID NO: 14 - DNA, 22 bp, synthetic construct
  source 1..22: mol_type = other DNA
tctcgatgagctgatgctttgg

SEQ ID NO: 15 - DNA, 21 bp, synthetic construct
  source 1..21: mol_type = other DNA
ggaagcacaacaccgaaattg

SEQ ID NO: 16 - DNA, 22 bp, synthetic construct
  source 1..22: mol_type = other DNA
caccgatatcctcgactgaaac

SEQ ID NO: 17 - DNA, 18 bp, synthetic construct
  source 1..18: mol_type = other DNA
atggtgttggtcgttgct

SEQ ID NO: 18 - DNA, 21 bp, synthetic construct
  source 1..21: mol_type = other DNA
cccgatctagtaacatagatg

SEQ ID NO: 19 - DNA, 13490 bp, synthetic construct
  source 1..13490: mol_type = other DNA
tggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacagatccggtcggcatctactctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgcacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgccccccgggatctgcgaaagctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattcgagctcggtacccggggatcctctagagtcgacctgcaggcatgcaagcttagctagcatactcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttaaaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgagctcctcccccctccccctccgccgccgccgcgccggtaaccaccccgcccctctcctctttctttctccgttttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaagccatggccggaaaggatgaggcggaagggctcgaggcgaggctgctgctgctgccgcctgaggcggcggcggaggagccgacgaggtgtggcggcggcgacggcggcggcggcgggaggaagcggaagaagacgtaccttgatgtccttggcgtgtgctgctccgcggaggtcgcgctggtggagcggctgctcgcgccgctcgacggcgtccgcgtggtgtccgtcgtcgtggcgtcccgcaccgtcgtcgtcgagcacgaccccgccgccgccccggagtccgccatcgtgaaggcgctgaacaaggccggcctcgaggcgtcggtgcgagcctacggcagcagcggcgtggtcagccggtggccgagcccgtacatcgtcgcctccggcgtcctcctcacggcgtccttcttcgagtggctcttccctcccctccagtgcctggccgtggcggccgtcgtcgccggcgcgccgccgatggtgcgccgtgggttcgccgcggcgagccggctgtcgctcgacatcaacgtcctcatgctcatcgccgtcgccggcgcgctctgcctcggcgactacacggaggccggcgccatcgtcttcctcttcaccaccgcagaatggctcgagacgctggcctgcacaaaggcgagcgccgggatgtcgtcgttgatgggcatgctgccggtgaaggcggtcatcgcgacgacgggcgaggttgtcagcgtgcgcgacgtccgcgtgggcgacgtcgtcgcggtcagggccggcgagatcgtccccgtggacggcgtggtggtggacggccagagcgaggtggacgagaggagcctcaccggcgagtccttcccggtgccgaagcagccgcactccgaggtctgggccggcacaatgaactttgacggttacattgctgtgagaactacggctctcgccgagaactcgacggtggcgaagatggagaggctggtggaggcggcgcagaacagcaggtcgaagacgcagcggctgatcgattcgtgcgcaaagtactacacgccggccgtggtggttgttgcagcaggagtggccctgatcccggctctgctcggagcagatggccttgagcaatggtggaagctggctctggtgatgcttgtgagcgcgtgcccctgcgcattagtgctgtccacaccggtggcatccttctgcgcaatgctgcgcgctgcgaggatggggatcttcatcaagggtggagatgttcttgaatcacttggggagatcagggccgtcgcgttcgacaagaccgggacgatcaccagaggagagttcagcatcgactcgttccatctggttggggatcacaaggttgagatggatcatcttctttactggattgcaagcattgagagcaagtcaagccacccaatggcagctgcacttgtggagtatgctcaatccaaatccatccaaccaaacccggaaaatgtgggcgattttcggatatatcccggggaggggatctatggagagattcatggaaagcacatctacattgggaacagaaggacattggcaagagcgtcatcacctcagtcaactcaagaaatgggtgagatgatcaagggcgtgtcgatcggctacgtgatctgcgacggcgagctcgccggcgtcttctcgctctccgacgactgccggaccggcgcggcggaggcgatccgggagcttggatcgctgggcatcaagtcggttatgctgaccggggacagcagcgcggcggcgacgcacgcgcagggccagctcggcggcgtcatggaggagctccactccgagctcctcccggaggacaaggtccggctcgtcagcggcctcaaggcgaggttcggcccgacgatgatggtcggggacgggatgaacgacgcggcggcgctggcggcggcggacgtgggcgtgtcgatgggcatctccgggtcggcggcggcgatggagaccagccacgcgacgctcatgtcgagcgacgtgctcagggtccccgaggccgtcaggctcggccggtgcgcccgccggaccatcgccgtcaacgtggccggctcggtggccgtgaaggccgccgtgctcgcgctggccgcggcgtggcgccccgtgctctgggccgccgtgctcgccgacgtcgggacgtgcctcctcgtcgtgctcaacagcatgacgctgctcagggaggagtggaagggcggcgccaaggaggacggcgcgtgccgcgccacggcgaggtcgctggtgatgaggtcccagctcgccgccgactcccaagcacccaacgccgcagacgctggcgccgccggccgtgagcaaacgaacggctgccgttgctgtccgaagccgggcatgtcgcccgagcactcggttgtcatcgacatccgagccgacggcgagcgtcaagaagagcggccggccgaggcggccgtcgttgccaaatgttgcggcggaggcggcggcgagggcatccgctgcggagcctccaagaagccaactgcaacggtcgttgttgccaaatgttgcgggggcggcggcggcggcgagggcaccaggtgcggcgcgtccaagaatccggcaacggcggccgtcgttgccaaatgttgcagcggaggcggcggcgagggcatcggctgcggagcctccaagaagccaactgcaacggccgttgttgccaaatgttgcggcggcggcggcgagggcaccaggtgcgccgcgtccaagaagccggcaacggcggccgtcgttgccaaatgttgcggtggcgatggcggcgagggcaccggatgtggtgcctccaagaggtcgccgccggctgagggaagctgctccggcggtgaaggcggtaccaatggtgttggtcgttgctgcacgagcgtgaagaggccaacctgttgcgacatgggagcggcggaggtgtccgattcttcgccggagacggcgaaagactgtagaaatgggaggtgttgcgcgaagacgatgaactccggtgaagtgaaaggatgaggtgacccaagcttgtgacggcaaagtaaggtgaccagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctgg

SEQ ID NO: 20 - DNA, 7237 bp, synthetic construct
  source 1..7237: mol_type = other DNA
tggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacagatccggtcggcatctactctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgcacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgccccccgggatctgcgaaagctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattcgagctcggtacccggggatcctctagagtcgacctgcaggcatgcaagcttagctagcatactcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttaaaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgagctcctcccccctccccctccgccgccgccgcgccggtaaccaccccgcccctctcctctttctttctccgttttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaagccatggccggaaaggatgaggcggaagggctcgaggcgaggctgctgctgctgccgcctgaggcggcggcggaggagccgacgaggtgtggcggcggcgacggcggcggcggcgggaggaagcggaagaagacgtaccttgatgtccttggcgtgtgctgctccgcggaggtcgcgctggtggagcggctgctcgcgccgctcgacggcgtccgcgtggtgtccgtcgtcgtggcgtcccgcaccgtcgtcgtcgagcacgaccccgccgccgccccggagtccgccatcgtgaaggcgctgaacaaggccggcctcgaggcgtcggtgcgagcctacggcagcagcggcgtggtcagccggtggccgagcccgtacatcgtcgcctccggcgtcctcctcacggcgtccttcttcgagtggctcttccctcccctccagtgcctggccgtggcggccgtcgtcgccggcgcgccgccgatggtgcgccgtgggttcgccgcggcgagccggctgtcgctcgacatcaacgtcctcatgctcatcgccgtcgccggcgcgctctgcctcggcgactacacggaggccggcgccatcgtcttcctcttcaccaccgcagaatggctcgagacgctggcctgcacaaaggcgagcgccgggatgtcgtcgttgatgggcatgctgccggtgaaggcggtcatcgcgacgacgggcgaggttgtcagcgtgcgcgacgtccgcgtgggcgacgtcgtcgcggtcagggccggcgagatcgtccccgtggacggcgtggtggtggacggccagagcgaggtggacgagaggagcctcaccggcgagtccttcccggtgccgaagcagccgcactccgaggtctgggccggcacaatgaactttgacggttacattgctgtgagaactacggctctcgccgagaactcgacggtggcgaagatggagaggctggtggaggcggcgcagaacagcaggtcgaagacgcagcggctgatcgattcgtgcgcaaagtactacacgccggccgtggtggttgttgcagcaggagtggccctgatcccggctctgctcggagcagatggccttgagcaatggtggaagctggctctggtgatgcttgtgagcgcgtgcccctgcgcattagtgctgtccacaccggtggcatccttctgcgcaatgctgcgcgctgcgaggatggggatcttcatcaagggtggagatgttcttgaatcacttggggagatcagggccgtcgcgttcgacaagaccgggacgatcaccagaggagagttcagcatcgactcgttccatctggttggggatcacaaggttgagatggatcatcttctttactggattgcaagcattgagagcaagtcaagccacccaatggcagctgcacttgtggagtatgctcaatccaaatccatccaaccaaacccggaaaatgtgggcgattttcggatatatcccggggaggggatctatggagagattcatggaaagcacatctacattgggaacagaaggacattggcaagagcgtcatcacctcagtcaactcaagaaatgggtgagatgatcaagggcgtgtcgatcggctacgtgatctgcgacggcgagctcgccggcgtcttctcgctctccgacgactgccggaccggcgcggcggaggcgatccgggagcttggatcgctgggcatcaagtcggttatgctgaccggggacagcagcgcggcggcgacgcacgcgcagggccagctcggcggcgtcatggaggagctccactccgagctcctcccggaggacaaggtccggctcgtcagcggcctcaaggcgaggttcggcccgacgatgatggtcggggacgggatgaacgacgcggcggcgctggcggcggcggacgtgggcgtgtcgatgggcatctccgggtcggcggcggcgatggagaccagccacgcgacgctcatgtcgagcgacgtgctcagggtccccgaggccgtcaggctcggccggtgcgcccgccggaccatcgccgtcaacgtggccggctcggtggccgtgaaggccgccgtgctcgcgctggccgcggcgtggcgccccgtgctctgggccgccgtgctcgccgacgtcgggacgtgcctcctcgtcgtgctcaacagcatgacgctgctcagggaggagtggaagggcggcgccaaggaggacggcgcgtgccgcgccacggcgaggtcgctggtgatgaggtcccagctcgccgccgactcccaagcacccaacgccgcagacgctggcgccgccggccgtgagcaaacgaacggctgccgttgctgtccgaagccgggcatgtcgcccgagcactcggttgtcatcgacatccgagccgacggcgagcgtcaagaagagcggccggccgaggcggccgtcgttgccaaatgttgcggcggaggcggcggcgagggcatccgctgcggagcctccaagaagccaactgcaacggtcgttgttgccaaatgttgcgggggcggcggcggcggcgagggcaccaggtgcggcgcgtccaagaatccggcaacggcggccgtcgttgccaaatgttgcagcggaggcggcggcgagggcatcggctgcggagcctccaagaagccaactgcaacggccgttgttgccaaatgttgcggcggcggcggcgagggcaccaggtgcgccgcgtccaagaagccggcaacggcggccgtcgttgccaaatgttgcggtggcgatggcggcgagggcaccggatgtggtgcctccaagaggtcgccgccggctgagggaagctgctccggcggtgaaggcggtaccaatggtgttggtcgttgctgcacgagcgtgaagaggccaacctgttgcgacatgggagcggcggaggtgtccgattcttcgccggagacggcgaaagactgtagaaatgggaggtgttgcgcgaagacgatgaactccggtgaagtgaaaggatgaggtgacccaagcttgtgacggcaaagtaaggtgaccagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattaaactatcagtgtttgacaggatatattggcgggtaaac

SEQ ID NO: 21 - DNA, 1419 bp, synthetic construct
  source 1..1419: mol_type = other DNA
agcttagctagcatactcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttaaaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgagctcctcccccctccccctccgccgccgccgcgccggtaaccaccccgcccctctcctctttctttctccgttttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaagc

SEQ ID NO: 22 - DNA, 3015 bp, synthetic construct
  source 1..3015: mol_type = other DNA
atggccggaaaggatgaggcggaagggctcgaggcgaggctgctgctgctgccgcctgaggcggcggcggaggagccgacgaggtgtggcggcggcgacggcggcggcggcgggaggaagcggaagaagacgtaccttgatgtccttggcgtgtgctgctccgcggaggtcgcgctggtggagcggctgctcgcgccgctcgacggcgtccgcgtggtgtccgtcgtcgtggcgtcccgcaccgtcgtcgtcgagcacgaccccgccgccgccccggagtccgccatcgtgaaggcgctgaacaaggccggcctcgaggcgtcggtgcgagcctacggcagcagcggcgtggtcagccggtggccgagcccgtacatcgtcgcctccggcgtcctcctcacggcgtccttcttcgagtggctcttccctcccctccagtgcctggccgtggcggccgtcgtcgccggcgcgccgccgatggtgcgccgtgggttcgccgcggcgagccggctgtcgctcgacatcaacgtcctcatgctcatcgccgtcgccggcgcgctctgcctcggcgactacacggaggccggcgccatcgtcttcctcttcaccaccgcagaatggctcgagacgctggcctgcacaaaggcgagcgccgggatgtcgtcgttgatgggcatgctgccggtgaaggcggtcatcgcgacgacgggcgaggttgtcagcgtgcgcgacgtccgcgtgggcgacgtcgtcgcggtcagggccggcgagatcgtccccgtggacggcgtggtggtggacggccagagcgaggtggacgagaggagcctcaccggcgagtccttcccggtgccgaagcagccgcactccgaggtctgggccggcacaatgaactttgacggttacattgctgtgagaactacggctctcgccgagaactcgacggtggcgaagatggagaggctggtggaggcggcgcagaacagcaggtcgaagacgcagcggctgatcgattcgtgcgcaaagtactacacgccggccgtggtggttgttgcagcaggagtggccctgatcccggctctgctcggagcagatggccttgagcaatggtggaagctggctctggtgatgcttgtgagcgcgtgcccctgcgcattagtgctgtccacaccggtggcatccttctgcgcaatgctgcgcgctgcgaggatggggatcttcatcaagggtggagatgttcttgaatcacttggggagatcagggccgtcgcgttcgacaagaccgggacgatcaccagaggagagttcagcatcgactcgttccatctggttggggatcacaaggttgagatggatcatcttctttactggattgcaagcattgagagcaagtcaagccacccaatggcagctgcacttgtggagtatgctcaatccaaatccatccaaccaaacccggaaaatgtgggcgattttcggatatatcccggggaggggatctatggagagattcatggaaagcacatctacattgggaacagaaggacattggcaagagcgtcatcacctcagtcaactcaagaaatgggtgagatgatcaagggcgtgtcgatcggctacgtgatctgcgacggcgagctcgccggcgtcttctcgctctccgacgactgccggaccggcgcggcggaggcgatccgggagcttggatcgctgggcatcaagtcggttatgctgaccggggacagcagcgcggcggcgacgcacgcgcagggccagctcggcggcgtcatggaggagctccactccgagctcctcccggaggacaaggtccggctcgtcagcggcctcaaggcgaggttcggcccgacgatgatggtcggggacgggatgaacgacgcggcggcgctggcggcggcggacgtgggcgtgtcgatgggcatctccgggtcggcggcggcgatggagaccagccacgcgacgctcatgtcgagcgacgtgctcagggtccccgaggccgtcaggctcggccggtgcgcccgccggaccatcgccgtcaacgtggccggctcggtggccgtgaaggccgccgtgctcgcgctggccgcggcgtggcgccccgtgctctgggccgccgtgctcgccgacgtcgggacgtgcctcctcgtcgtgctcaacagcatgacgctgctcagggaggagtggaagggcggcgccaaggaggacggcgcgtgccgcgccacggcgaggtcgctggtgatgaggtcccagctcgccgccgactcccaagcacccaacgccgcagacgctggcgccgccggccgtgagcaaacgaacggctgccgttgctgtccgaagccgggcatgtcgcccgagcactcggttgtcatcgacatccgagccgacggcgagcgtcaagaagagcggccggccgaggcggccgtcgttgccaaatgttgcggcggaggcggcggcgagggcatccgctgcggagcctccaagaagccaactgcaacggtcgttgttgccaaatgttgcgggggcggcggcggcggcgagggcaccaggtgcggcgcgtccaagaatccggcaacggcggccgtcgttgccaaatgttgcagcggaggcggcggcgagggcatcggctgcggagcctccaagaagccaactgcaacggccgttgttgccaaatgttgcggcggcggcggcgagggcaccaggtgcgccgcgtccaagaagccggcaacggcggccgtcgttgccaaatgttgcggtggcgatggcggcgagggcaccggatgtggtgcctccaagaggtcgccgccggctgagggaagctgctccggcggtgaaggcggtaccaatggtgttggtcgttgctgcacgagcgtgaagaggccaacctgttgcgacatgggagcggcggaggtgtccgattcttcgccggagacggcgaaagactgtagaaatgggaggtgttgcgcgaagacgatgaactccggtgaagtgaaaggatga

SEQ ID NO: 23 - DNA, 253 bp, synthetic construct
  source 1..253: mol_type = other DNA
cgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggg

SEQ ID NO: 24 - DNA, 781 bp, synthetic construct
  source 1..781: mol_type = other DNA
agagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccat

SEQ ID NO: 25 - DNA, 1026 bp, synthetic construct
  source 1..1026: mol_type = other DNA
ctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgcacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcat

SEQ ID NO: 26 - DNA, 185 bp, synthetic construct
  source 1..185: mol_type = other DNA
taattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaa

SEQ ID NO: 27 - DNA, 25 bp, synthetic construct
  source 1..25: mol_type = other DNA
tggcaggatatattgtggtgtaaac

SEQ ID NO: 28 - DNA, 25 bp, synthetic construct
  source 1..25: mol_type = other DNA
tgacaggatatattggcgggtaaac

SEQ ID NO: 29 - DNA, 12002 bp, synthetic construct
  source 1..12002: mol_type = other DNA
tgacaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacagatccggtcggcatctactctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgcacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgccccccgggatctgcgaaagctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattcgagctcggtacccggggatcctctagatagctagcatactcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttaaaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgagctcctcccccctccccctccgccgccgccgcgccggtaaccaccccgcccctctcctctttctttctccgttttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaagccatggcgtctaaaccaagcagccgagcggaaagcaaccaggcggcggcggcggtgccgtcgctgtacgggcgcgccctgccgtcgccgccggcggtggagttcgcgtcggcggaggggcggcggctgttcgcggaggcgctccaggggggcaccatgcaggggttcttcagcctcgtctccgtcttccagacgcagtcggagccggccttctgcggcctcgccacgctcgccgtcgtcctcaacgcgctccgcatcgacccggggcggcggtggaagggcccctggcggtggttcgacgagtccatgctcgactgctgcgagcacctcgacacggtcagggcggaggggatcaccttcggcaaggtcgcctgcctcgcccactgctccggcgccgacgtccgcaccttccgcgccgcccaggccacgctcgccgacctccgccgccacctcctgcgctgcgcctcctcccaggattgccatctcgtcgcctcctaccaccggaaacttctcggccagactggaacagggcatttctcgccgattggcggctaccatgctgggcaggacatggcgctgatcctggatgtcgctcgcttcaaataccctcctcattggattccgctgccgcttctttgggaagccatgaacacgattgatgaagcaactgggcttctcagggggttcatgcttatctcaaggaatactgaagctcctttattgatccgtgcagtgaattgcagggatgaaagctggcaaagcatggcaaagtattgcatagaagttgtcccaaatcttttgagggataacagcgtagacaatgtcctaacaattctttcccgtttagtgaatcatcttcctcccaatgctggaaattttatcaaatgggtcattgaagttaggagacaagaggaaggaggatccagcccaagtaaagaggctaacgaaatgcctttcctgaaggaaaaggtcctacagcaaatccgtgatactaaactttttcagctggtccacaaactgcaatgttctaagcagccctgttgtagttgctcgtctttaacggacgaagattccatttcccagattgcagccagtgtgtgctgtgaagcaaccgcattgctaagtgggaatctgtcatcaagagatggattatttttcagtgaaacttgttccggatgtacacaagtgaatgatgaggggcttaaaaatgtcattacaggcaaagtggtatctgaaggcaatggacatgttgataaactttcaccgatatcctcgactgaaacatgcttctgcaattcaactctgagcaatgaaactgtcaattatccatcaaacacagacattctaactgttctattactgtcgttacatcctagcacatggttgtgcattgaagatgagaagttgaaagctgaatttcagagccttgtttctacggacgatcttcctgatcctcttaaactggagatattgcacctaaggaggcagctccgttatctgaaggcttgtagagaaaaggaggcatatgaagatacttggccacaaccttgggaacaatgctaagcttgtgacggcaaagtaaggtgaccagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctgg

SEQ ID NO: 30 - DNA, 5749 bp, synthetic construct
  source 1..5749: mol_type = other DNA
tggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacagatccggtcggcatctactctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgcacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgccccccgggatctgcgaaagctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattcgagctcggtacccggggatcctctagatagctagcatactcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttaaaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgagctcctcccccctccccctccgccgccgccgcgccggtaaccaccccgcccctctcctctttctttctccgttttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaagccatggcgtctaaaccaagcagccgagcggaaagcaaccaggcggcggcggcggtgccgtcgctgtacgggcgcgccctgccgtcgccgccggcggtggagttcgcgtcggcggaggggcggcggctgttcgcggaggcgctccaggggggcaccatgcaggggttcttcagcctcgtctccgtcttccagacgcagtcggagccggccttctgcggcctcgccacgctcgccgtcgtcctcaacgcgctccgcatcgacccggggcggcggtggaagggcccctggcggtggttcgacgagtccatgctcgactgctgcgagcacctcgacacggtcagggcggaggggatcaccttcggcaaggtcgcctgcctcgcccactgctccggcgccgacgtccgcaccttccgcgccgcccaggccacgctcgccgacctccgccgccacctcctgcgctgcgcctcctcccaggattgccatctcgtcgcctcctaccaccggaaacttctcggccagactggaacagggcatttctcgccgattggcggctaccatgctgggcaggacatggcgctgatcctggatgtcgctcgcttcaaataccctcctcattggattccgctgccgcttctttgggaagccatgaacacgattgatgaagcaactgggcttctcagggggttcatgcttatctcaaggaatactgaagctcctttattgatccgtgcagtgaattgcagggatgaaagctggcaaagcatggcaaagtattgcatagaagttgtcccaaatcttttgagggataacagcgtagacaatgtcctaacaattctttcccgtttagtgaatcatcttcctcccaatgctggaaattttatcaaatgggtcattgaagttaggagacaagaggaaggaggatccagcccaagtaaagaggctaacgaaatgcctttcctgaaggaaaaggtcctacagcaaatccgtgatactaaactttttcagctggtccacaaactgcaatgttctaagcagccctgttgtagttgctcgtctttaacggacgaagattccatttcccagattgcagccagtgtgtgctgtgaagcaaccgcattgctaagtgggaatctgtcatcaagagatggattatttttcagtgaaacttgttccggatgtacacaagtgaatgatgaggggcttaaaaatgtcattacaggcaaagtggtatctgaaggcaatggacatgttgataaactttcaccgatatcctcgactgaaacatgcttctgcaattcaactctgagcaatgaaactgtcaattatccatcaaacacagacattctaactgttctattactgtcgttacatcctagcacatggttgtgcattgaagatgagaagttgaaagctgaatttcagagccttgtttctacggacgatcttcctgatcctcttaaactggagatattgcacctaaggaggcagctccgttatctgaaggcttgtagagaaaaggaggcatatgaagatacttggccacaaccttgggaacaatgctaagcttgtgacggcaaagtaaggtgaccagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattaaactatcagtgtttgacaggatatattggcgggtaaac

SEQ ID NO: 31 - DNA, 1420 bp, synthetic construct
  source 1..1420: mol_type = other DNA
ctagatagctagcatactcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttaaaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgagctcctcccccctccccctccgccgccgccgcgccggtaaccaccccgcccctctcctctttctttctccgttttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaagc

SEQ ID NO: 32 - DNA, 1560 bp, synthetic construct
  source 1..1560: mol_type = other DNA
atggcgtctaaaccaagcagccgagcggaaagcaaccaggcggcggcggcggtgccgtcgctgtacgggcgcgccctgccgtcgccgccggcggtggagttcgcgtcggcggaggggcggcggctgttcgcggaggcgctccaggggggcaccatgcaggggttcttcagcctcgtctccgtcttccagacgcagtcggagccggccttctgcggcctcgccacgctcgccgtcgtcctcaacgcgctccgcatcgacccggggcggcggtggaagggcccctggcggtggttcgacgagtccatgctcgactgctgcgagcacctcgacacggtcagggcggaggggatcaccttcggcaaggtcgcctgcctcgcccactgctccggcgccgacgtccgcaccttccgcgccgcccaggccacgctcgccgacctccgccgccacctcctgcgctgcgcctcctcccaggattgccatctcgtcgcctcctaccaccggaaacttctcggccagactggaacagggcatttctcgccgattggcggctaccatgctgggcaggacatggcgctgatcctggatgtcgctcgcttcaaataccctcctcattggattccgctgccgcttctttgggaagccatgaacacgattgatgaagcaactgggcttctcagggggttcatgcttatctcaaggaatactgaagctcctttattgatccgtgcagtgaattgcagggatgaaagctggcaaagcatggcaaagtattgcatagaagttgtcccaaatcttttgagggataacagcgtagacaatgtcctaacaattctttcccgtttagtgaatcatcttcctcccaatgctggaaattttatcaaatgggtcattgaagttaggagacaagaggaaggaggatccagcccaagtaaagaggctaacgaaatgcctttcctgaaggaaaaggtcctacagcaaatccgtgatactaaactttttcagctggtccacaaactgcaatgttctaagcagccctgttgtagttgctcgtctttaacggacgaagattccatttcccagattgcagccagtgtgtgctgtgaagcaaccgcattgctaagtgggaatctgtcatcaagagatggattatttttcagtgaaacttgttccggatgtacacaagtgaatgatgaggggcttaaaaatgtcattacaggcaaagtggtatctgaaggcaatggacatgttgataaactttcaccgatatcctcgactgaaacatgcttctgcaattcaactctgagcaatgaaactgtcaattatccatcaaacacagacattctaactgttctattactgtcgttacatcctagcacatggttgtgcattgaagatgagaagttgaaagctgaatttcagagccttgtttctacggacgatcttcctgatcctcttaaactggagatattgcacctaaggaggcagctccgttatctgaaggcttgtagagaaaaggaggcatatgaagatacttggccacaaccttgggaacaatgctaa

SEQ ID NO: 33 - DNA, 21487 bp, synthetic construct
  source 1..21487: mol_type = other DNA
tggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacagatccggtcggcatctactctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgcacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgccccccgggatctgcgaaagctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctctagatagctagcatactcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttaaaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgagctcctcccccctccccctccgccgccgccgcgccggtaaccaccccgcccctctcctctttctttctccgttttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaagccatgggttttgatccactggaatggtactgccagccggtcaatggcggggtgtggtcagatgtagagaatgcttttggtgcatacacaccatgtggcactgaaaccttggtggtgtgcatctcatactttgcactcttcggtgtttgcttctatcggatatggaggacgacgagggactacacggtgcagcgctacaaattgcggtcgccatactacaactatatgctcggcctgcttgtggtgttgtgcatagctgaatcactgtatcggatcgctactggcacctccataatgaacttggatggggagactagccttgctccatttgaggtgagaacatgtcccaagtataagtgtatcagcacgtagagttaagcacacttgttcagaattttacttctaccgaattcttgcatcccttgaccttttcaatcacgtaattctgcctctacaggttacctcatctatcattgagattgctgcttggtgttgtatgcttgtaatgattgcacttgagacaagaatttacatctatgagtttagatggtacatccggtttgtggtcatttacatcttggttggggaagctgctatgttcaatcttgtgctttcagtaaggcagtattatagttcgaggtatagtacacatgagacatgctttattcatgaaatttattgaatgttgattgatagagaactttatactctaaatttttcaatgaagactgacttctgtattttggttatgcagttcaatattttacttgtactgcagtgagataatattcaaggtttgcattcttgttatcttaataatcgtactgtagtgatatttgtcgtatactgaacttatcatctctgcagctcttgtttggaattctcatggtggtttatctgcctagcttggattcctatccgggttatactccagtcaggcatgaggcacttgttgataatactgattacgaacctctgcctggtggggagcagatttgccctgagaggcatgctaatatattttccagtgagtaaatataataagttgtgcggttgtgccacactattgcatacatccataatgttttgtttgtcctcataatttctctaaatttctagaagcacactgaggctttttggttgtcttaccttgctagggatattcttttcatggatgactcctctaatgcaacaaggatttaaaaggcccatcaccgataaggatatttggaaattagacagttgggatgagactgaaacattgtacaaccggtatgttctgcaaccatttttactgaaatgagaaacacttgtatatgcagatcttaaccttataatctttcctcaggttccaaaaatgctggaacaatgagcttcaaaaaccaaagccttggctgttacgggctctgcatagcagccttggtggaaggtatatttgtacagatgatagctatattttccagtaaacctttagggtggtcccttcttgctacattgtttgtgttcacgtagtgattggtttcattcattttcaggttttggctaggaggattttttaaggtaggtaaatgtatctcgacttttgtaaagttatccttcaatttcgtttgcatttttgtttgatggcaagcattttgtgttattcctggcaactcatcctttttcctccaaaattttgtagattggcaatgatgcttctcagtttgttggcccactcatattgaacctcttgttggaggtatgcttctagtttcatgaaccaatggcaataaaattgcattgcattacctttacaagccttattctactattgtttttaccatcaacaaacttgtgaagtaattcaaaacatactgggtaattgtgcagattttaatatttatatatgaaagaatagatccaacttttctcttatgttgttgagatgctagtgttaggtgctttgtaccactaaaggtcaatgttcttttggcagtctatgcaaaaaggtgatccatcttggagcggttacatctatgctttctcaatcttcgctggagtggtatggattattttcttctattgcatgtttcacacttatatgtcttgttgattcagtgtatttgaacttccaagcaacaattatgctgcatgtgagccaaaactggacagacttttgatcacttaccttttgtcgaccattctgcagtcactgggtgttctttctgaagcacagtactttcagaatgtcatgcgaacgggtttcaggttgaggtcaacattggtaggtgttgcttgtttagcatggtttaatatgatgtcaacaggcaaccagcaaaccacttgttccactatttttctggaccactcttttttttttctctactcccatggttatcttgagcattgaacttcctgatttatgcatcaaggacatgtgtaattgtaatattaagtttagacctttatttttcttatgttcaccatgatataatattatctggtaataattgactttcctgcaagtaacactaccatagccatctcagctgctgttatatgtttagttacacgcttatgccatgattctgttgtttcgcagatcgcagctgttttccgtaagtctttgcgactaactaatgatagtcgaaagaagtttgcctctgggaggataaccaatttgatttcaactgacgcagaatctcttcaggtgtgattttatttcataggtttaatcagtgttacccagttcttcaacattattgattttaaatattgtttggcaacataaaattagataaaaaaccaaatgatatgttagataccttgttgatgtctgaattgataagtaaatggaaaaccgtggaaatgttttgttaggaaagagcaatggagcaagaaatacaatatgtgctacaatatggagctatggttatttatattttatgttgtacatttggtttgtactttttttacttttgcagcaagtgtgccagcaacttcacagtctatggtctgctcctttccgcattgttattgccatggtccttctatacgcacaactaggccctgctgcattggtcggtgcagccatgttggttcttttgttcccaattcaggtagatttctctttagacattcaattatggaaggtcttatgttgcatttttaactttctatctggtccctacattaccaatatctactttgtgttttttatattttgattctttttagggaatgtgcagaaagattgtactgatatatcgattcttatatggagcctagagttcacattagagtctttcatgccatcatttagttgttgattctacttcctgttatctatcctgaggttgtattgtagtactgtttgtacgtctgcttagttttctgccataccccgtaagaaattctttgatgttgtccataaattggagttgatcaagtcaatgcttaaatccctaatatttggctaatccatattgctcatgctttctcttttgaagcttataatgcaaattgccagggcacttaacagttgctgattttcagattatgctatttttatgaatgatatatgcatttcaaataacgattttacttttaagcaaacttttgctactttcaacatagatctgactgttaacattttctttatttaatttaccagactgttatcataagcaaaatgcaaaaacttaccaaggaagggttgcaaaggactgacaggagaatcagtctaatgaatgaaatattagctgcaatggatacggtcaagtatgctttacatttacttgtgtttttactagaagtagtactaaaattttggtataaactgttccaaacattttcttggttagatgttatgcttgggagcaaagtttccagtcaaaagtgcaggacatccgtgatgatgaaatttcatggttccgcagcgctcaattgcttgctgcggtatgtccattttgtatttaagttatctttcacttgtgtcaatttcattctgctatatatgatctcttttttaatcgtgcatccatgaactctccttattacacaataagttttggagttgcatgctatagtatcgattgtactggtgcttcctggtcctataaagtttgtttacaataatgtacttagttactcaaacttaaaatagatatgagaatgagttgttgcatcccttattgtcggctcactattatcttttgtatttttccttgtctgctgcagctgaatagcttcatcctaaacagtatccccgttattgtcactgtggtttcatttggtgtatactctctgctgggaggtgacttgacaccagcaaaggcgttcacatcactttcactgtttgctgtcttaagatttccactttttatgctgccaaatctgataactcaggtgtgcaaaaagctgtaaattatgtcttgttaagtttcccactttctgtacaagttaagaagcatatcattttctgaacttatatgtttggattgataatcattcttgtaccagttaattttcatctgtctttggcagagaaaattagtacaatggtttttacaattttggtcacagtccaatgctggcttgagatagttctctgcttttcttgttcaacgactcttttgttagtactcatagtcaccttactattttttttccttttaaatttgacaggtggttaattgtaaagtgtcattgaaacgtctggaagatctcctcttggctgaagagagattacttctgcccaatccacctcttgatcctgagcttccagcaatttctattaagaatggatatttttcatgggaatcgcaggtaatttcatgtttcagatatttgcctggtatctaaatatatctatgttgactatcacacattcaactctcatcattcattgatattataatataacacatatattattgttctgttgtctggagtactgaaaagaaacaaaagtttgggcaaataaatactgaattagagtattgatagactagcatgcttcatgaatggcaaggtcagtgcaaaagagagcatctagttactgaggtactgaggtcgatccatagaaagatgtctattacagatgtctccgtagttcataatgaattgatgatttggtagtaagcttcacaatactgttctgaccagctcttattggtttccatttttgtcacataattttatacagttgttaacactgcatgtttcctctaatcttatgatgttgctccaaaaactaatttttcaaattttggcaggctgagagaccaacattatcaaacgtcaatctggatgtacctatgggcagtttggttgcaatagtaggaagcactggggaggggaagacttctcttatttctgcaatgcttggtgaaataccaccagtgtcaggatcgaatacatcggtggtccttcgtggaacagtggcttatgttcctcaagtttcatggatcttcaatgcaactgtgagacacaatatctccatttatgttgattgttatattgaaccttttatggcttctacagatataatgttatcatctctgcaggtccgggacaatatattgtttgggtctcccttccaacctccacgctacgagaaagcaatagatgtcacttcattacggcatgaccttgacttactcccagtaagttctaactatttcagtcttcagagatcgttctggaaatttttgacactcgagctttcatttgatagataacttgctggggagtgtgttttttttttcatttgtcatgttgaaattatgttgccaatacctttttctatcttggttttgcgctttatattcttgtacctgcaaattgacaagaaaatatatcttcttatgcttgttgtcagggtggtgacctaacagagattggagaaagaggagttaacattagtgggggacaaaaacagagagtctcaatggcaagagctgtttattctgattcagatgtctacatatttgatgatccactgagtgcattagatgcccatgttggtcgacaggtgcattcatcttgctaccactgcggttcatgctttcaattttacagtctcccccgcattatcaaatctgccagtggcagttggtaatggtgtcaatgttttgtggatatcagcatcttttttgtcgaaattcctttttactgagattcccttgtgtctgttttacttccctgggacatgaaaacatgaggttctatgccattggtttggcatgtgtcagccatgtttctgttatgaacatcacattgctatgcatttttttcttatgcccgtttactgcagataggcacactttctggttatagagtaattgcgtcgataagatatggtacaccatttgtcacggtagatacttaaatctgtttgttgtgaccgaactaatgttgtgaataattatatgccaaatcatcctatatgctgtgctttggtagcctgctacaagtgatgtaccttggaggctgtttggatccctcttaccaaaactagattttgttaaacagattttagatcctgacaaattgttatgtgatccaaacaaagattttcttctgtatgaaaatatttcagttcaactaaataatcttaattactgatgccctattatcacatgcttatgcaggtatttgacaaatgtattaaagaagagctacagcacaaaactagggttcttgttaccaatcagctgcattttctaccatatgtggataaaatactggtagtccatgatggtgtaattaaagaagaaggtacctttgatgaacttagcaactctggggaactctttaagaagctcatggaaaatgctggaaagatggaagaacagatggaagagaaacaggacgaaagcaaaagacaggatgacataaaacaccctgaaaatgggggcagtgtaatagctgatggtgatatgcagaagagtcaggacacttctaataaaacaaaacaggggaaatctgttctgattaaacaagaagaaagggaaactggagttataagtgcaaaggtcctttcacggtaaaagattgttgtctcattgtatggtagtatatttcatgtataatacagatagaagaaaggaaacaggagagggagggaggcataatattaaacatatttatagatcatgatgacctggaatatctgcttacttaatacctttacctttgcagctacaaaaatgcattgggagggatctgggtggtatctgttctctttttttgctatgcactcactgaagttcttcgcatttcaagtagcacatggttgagcgtttggacagatcaaggttctacgaaaatccatggccctggttactacaatttaatctacggccttctttcttttgggcaggtacttgtaccatgattttcatcttcttttcttgattagaatttgaatttatccaattgatcacatgctggctgcaaaatatgacattgtgttgtttataggttctagtcactctaacgaattcttattggctgatcacatcaagtcttcgagcagccaagaggttgcatgatgccatgctccggtctatattaagagctcccatggtattttttcacaccaatccacttggacgaatcatcaacagattttcaaaggatttgggtgacattgacaggaatgtagccatctttgtgaatatgtttatggcacaaatatctcagttgctctcaacatttgttctcattggcattgttagcactatgtctctgtgggctatcatgccacttctgattttattttatgcagcttacctctattaccaggtacttttgtgacagctgacttttttaaaaaatatatattaaagattgtattgttagtcctaacttcttgaatcttgacatctcatacctgtcttttcccagactacgtctcgtgaggtaaagcgcctggattccattacaaggtctcctgtgtatgctcaattttcagaggcattgaatggtctgtctacaatccgtgcctacaaagcctatgatagaatggcaaacatcaatgggaaatcaatggacaacaacatcagattcactcttgtgaacatgagttcaaacagatggctagccatccgtctggaaacattgggtggcatcatgatatggttcacagcaacatttgctgtcatgcaaaaccaacgagcagaaaatcaaaaggcttttgcctccacaatgggtcttcttcttacttacaccctcaatatcaccaatttgctcacggctgttcttcgtcttgctagtctggctgaaaacagtttaaatgctgttgagcgggtaggaacatacattgagttgccttctgaggctcctcctgtcattgaggatagcaggccacctcctggttggccatcatctggtgttgtcaagtttgaagacgtcgtgcttcggtaccgaccagaacttcctcctgttcttcatggcatatctttcatcattaatggcagcgaaaaggtaggaatagttggcagaacaggagctggtaaatctagcatgctgaatgctctgttccgtattgtggaactggaacgagggagaatattggttgatgattgtgacacttctaaatttggaatttgggacctacgaaaagtactaggaataataccacaggcacctgtcctattttcaggtttgtttgtgaaaatacatgatatatcttctcgtttgcttttcttacatcataaatgttttttttttttggtactattgtccaggttctgttcgatttaatctggatccttttaatgagcacaatgatgcggatctctgggaagcgcttgaaagggctcatctaaaagatgtcataaggaggaatgctctgggactagatgctgaggtacgtagtttgtttatcctttatttagttcagaccattatttcacttttttttaagacgatgtatgcctgtatagacttgtcagttttttttatgtcagtctatagtttagcagtttaccttttggtattcaatatattccaagagtttgctttcggaataaaacaggattgttatagaattatgctactgctgctttctccttgtagttaatacacaatattgaataaactttctttatagtagttaccttgttttttttaaacaaacacttcggaaaatttctgtggaacttcatcttgttaacacactcaggtctctgaggctggtgaaaattttagcgttggacagcggcagctactgagtttagctcgtgcattgctgcggagggcaaagatacttgttcttgatgaggcaacagcagctgttgatgttcgaacggatgctcttatacagaagacaatcagggaagaattcaagagttgtacaatgcttataattgctcaccgcctgaacactgttattgactgtgacaggttgcttattctaagtgctgggaaggtatgcttttgaagagatgtttcttcttttgttctggtgctttgcctctcatttgaatagtacaacaataaattcctgataatatcaacatgcttgataggtgtaatttccaaagtaaccaatgaaggattgttgcttgtctgttttggactcttgatttgaatgttgctttccttagctcctggagcttggtttccacaattttccaactgaactaaaacgtggtcatagtgagcttgtctgtaattagtatcatactatcctttacaatctctatattataaagtgttatttcaactgtcatgtttccagtgttctgttttcatattttattagtattatagatgacttgtaagagcattgaattcaccctgaaaagactcttggtatttattaacctagtgagatagatggcttgaaataattctgttcactctgacaaatagagagctaaacacgttcaggttgcactcttttcgtgctcagcctattatttttcttgattgcttaatttttggcacctcagttatgaaactactacttttcaggttttggagttcgactctcctgagaatcttttgagcaatgagcacagtgctttctccaagatggttcagagtacaggacctagcaatgcagagtacctgaaggtgtttttctatcatctcgatgttcttgaaatttgtgtactacagcataaaacagcgtttgataaataataatgagtgatgctgttcaatccatgttacactccatcgctcaagttttcacagttggctttcagcgaaattattgatatgtgttctgattcaacttttttttgactgaattattgacagactcttgtatttggggatggtgaggagaggttgcgaaaggaagaaagcaagatgcaagatatccagaggaagtgggtagcatctaaccgatgggctgttgctgcgcagtttgcgcttgccgctagccttgcctcttcacacagtgaccttcttgcactggaggctgctgagggaaacaacatcctcaggaaaacaaaggatgcagtaatcactctacagaatgtcctcgaagggaagcacaacaccgaaattgatgatacactcgctcagtatgaggttccatctgataggtggtggtcatcgctatacaaagtcatggaaggtacgttgatcacttcattttaacacctttctcttgtgcgatgcttcagagtagcatgtattgtgtgcaatataagtttattacactagtcccagtgtaaactggctaatattggtccgtcagagattctgatgaaaccaatagttaacagtgagtaccggattggtgcttggtcgatgcaaaagtccatatgttaacatgaagagaactgctgaaatataacgatagggataagattttcttggaccaacaaacatactaacacttgacaaaagcatgtcctccacatgccattgtcatgatctctttggtattcaaatctaaccctatcaatctgaccttttgatcaaatatgtatatctattttttttcaggacttgccatgatgagccgattgggtcgcaaccgtctacagcaaccaagttataattttgaaaacaacagttctattgactgggaccaaatgtagaagcttgtgacggcaaagtaaggtgaccagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctgg

SEQ ID NO: 34 - DNA, 15234 bp, synthetic construct
  source 1..15234: mol_type = other DNA
tggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacagatccggtcggcatctactctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgcacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgccccccgggatctgcgaaagctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctctagatagctagcatactcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttaaaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgagctcctcccccctccccctccgccgccgccgcgccggtaaccaccccgcccctctcctctttctttctccgttttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaagccatgggttttgatccactggaatggtactgccagccggtcaatggcggggtgtggtcagatgtagagaatgcttttggtgcatacacaccatgtggcactgaaaccttggtggtgtgcatctcatactttgcactcttcggtgtttgcttctatcggatatggaggacgacgagggactacacggtgcagcgctacaaattgcggtcgccatactacaactatatgctcggcctgcttgtggtgttgtgcatagctgaatcactgtatcggatcgctactggcacctccataatgaacttggatggggagactagccttgctccatttgaggtgagaacatgtcccaagtataagtgtatcagcacgtagagttaagcacacttgttcagaattttacttctaccgaattcttgcatcccttgaccttttcaatcacgtaattctgcctctacaggttacctcatctatcattgagattgctgcttggtgttgtatgcttgtaatgattgcacttgagacaagaatttacatctatgagtttagatggtacatccggtttgtggtcatttacatcttggttggggaagctgctatgttcaatcttgtgctttcagtaaggcagtattatagttcgaggtatagtacacatgagacatgctttattcatgaaatttattgaatgttgattgatagagaactttatactctaaatttttcaatgaagactgacttctgtattttggttatgcagttcaatattttacttgtactgcagtgagataatattcaaggtttgcattcttgttatcttaataatcgtactgtagtgatatttgtcgtatactgaacttatcatctctgcagctcttgtttggaattctcatggtggtttatctgcctagcttggattcctatccgggttatactccagtcaggcatgaggcacttgttgataatactgattacgaacctctgcctggtggggagcagatttgccctgagaggcatgctaatatattttccagtgagtaaatataataagttgtgcggttgtgccacactattgcatacatccataatgttttgtttgtcctcataatttctctaaatttctagaagcacactgaggctttttggttgtcttaccttgctagggatattcttttcatggatgactcctctaatgcaacaaggatttaaaaggcccatcaccgataaggatatttggaaattagacagttgggatgagactgaaacattgtacaaccggtatgttctgcaaccatttttactgaaatgagaaacacttgtatatgcagatcttaaccttataatctttcctcaggttccaaaaatgctggaacaatgagcttcaaaaaccaaagccttggctgttacgggctctgcatagcagccttggtggaaggtatatttgtacagatgatagctatattttccagtaaacctttagggtggtcccttcttgctacattgtttgtgttcacgtagtgattggtttcattcattttcaggttttggctaggaggattttttaaggtaggtaaatgtatctcgacttttgtaaagttatccttcaatttcgtttgcatttttgtttgatggcaagcattttgtgttattcctggcaactcatcctttttcctccaaaattttgtagattggcaatgatgcttctcagtttgttggcccactcatattgaacctcttgttggaggtatgcttctagtttcatgaaccaatggcaataaaattgcattgcattacctttacaagccttattctactattgtttttaccatcaacaaacttgtgaagtaattcaaaacatactgggtaattgtgcagattttaatatttatatatgaaagaatagatccaacttttctcttatgttgttgagatgctagtgttaggtgctttgtaccactaaaggtcaatgttcttttggcagtctatgcaaaaaggtgatccatcttggagcggttacatctatgctttctcaatcttcgctggagtggtatggattattttcttctattgcatgtttcacacttatatgtcttgttgattcagtgtatttgaacttccaagcaacaattatgctgcatgtgagccaaaactggacagacttttgatcacttaccttttgtcgaccattctgcagtcactgggtgttctttctgaagcacagtactttcagaatgtcatgcgaacgggtttcaggttgaggtcaacattggtaggtgttgcttgtttagcatggtttaatatgatgtcaacaggcaaccagcaaaccacttgttccactatttttctggaccactcttttttttttctctactcccatggttatcttgagcattgaacttcctgatttatgcatcaaggacatgtgtaattgtaatattaagtttagacctttatttttcttatgttcaccatgatataatattatctggtaataattgactttcctgcaagtaacactaccatagccatctcagctgctgttatatgtttagttacacgcttatgccatgattctgttgtttcgcagatcgcagctgttttccgtaagtctttgcgactaactaatgatagtcgaaagaagtttgcctctgggaggataaccaatttgatttcaactgacgcagaatctcttcaggtgtgattttatttcataggtttaatcagtgttacccagttcttcaacattattgattttaaatattgtttggcaacataaaattagataaaaaaccaaatgatatgttagataccttgttgatgtctgaattgataagtaaatggaaaaccgtggaaatgttttgttaggaaagagcaatggagcaagaaatacaatatgtgctacaatatggagctatggttatttatattttatgttgtacatttggtttgtactttttttacttttgcagcaagtgtgccagcaacttcacagtctatggtctgctcctttccgcattgttattgccatggtccttctatacgcacaactaggccctgctgcattggtcggtgcagccatgttggttcttttgttcccaattcaggtagatttctctttagacattcaattatggaaggtcttatgttgcatttttaactttctatctggtccctacattaccaatatctactttgtgttttttatattttgattctttttagggaatgtgcagaaagattgtactgatatatcgattcttatatggagcctagagttcacattagagtctttcatgccatcatttagttgttgattctacttcctgttatctatcctgaggttgtattgtagtactgtttgtacgtctgcttagttttctgccataccccgtaagaaattctttgatgttgtccataaattggagttgatcaagtcaatgcttaaatccctaatatttggctaatccatattgctcatgctttctcttttgaagcttataatgcaaattgccagggcacttaacagttgctgattttcagattatgctatttttatgaatgatatatgcatttcaaataacgattttacttttaagcaaacttttgctactttcaacatagatctgactgttaacattttctttatttaatttaccagactgttatcataagcaaaatgcaaaaacttaccaaggaagggttgcaaaggactgacaggagaatcagtctaatgaatgaaatattagctgcaatggatacggtcaagtatgctttacatttacttgtgtttttactagaagtagtactaaaattttggtataaactgttccaaacattttcttggttagatgttatgcttgggagcaaagtttccagtcaaaagtgcaggacatccgtgatgatgaaatttcatggttccgcagcgctcaattgcttgctgcggtatgtccattttgtatttaagttatctttcacttgtgtcaatttcattctgctatatatgatctcttttttaatcgtgcatccatgaactctccttattacacaataagttttggagttgcatgctatagtatcgattgtactggtgcttcctggtcctataaagtttgtttacaataatgtacttagttactcaaacttaaaatagatatgagaatgagttgttgcatcccttattgtcggctcactattatcttttgtatttttccttgtctgctgcagctgaatagcttcatcctaaacagtatccccgttattgtcactgtggtttcatttggtgtatactctctgctgggaggtgacttgacaccagcaaaggcgttcacatcactttcactgtttgctgtcttaagatttccactttttatgctgccaaatctgataactcaggtgtgcaaaaagctgtaaattatgtcttgttaagtttcccactttctgtacaagttaagaagcatatcattttctgaacttatatgtttggattgataatcattcttgtaccagttaattttcatctgtctttggcagagaaaattagtacaatggtttttacaattttggtcacagtccaatgctggcttgagatagttctctgcttttcttgttcaacgactcttttgttagtactcatagtcaccttactattttttttccttttaaatttgacaggtggttaattgtaaagtgtcattgaaacgtctggaagatctcctcttggctgaagagagattacttctgcccaatccacctcttgatcctgagcttccagcaatttctattaagaatggatatttttcatgggaatcgcaggtaatttcatgtttcagatatttgcctggtatctaaatatatctatgttgactatcacacattcaactctcatcattcattgatattataatataacacatatattattgttctgttgtctggagtactgaaaagaaacaaaagtttgggcaaataaatactgaattagagtattgatagactagcatgcttcatgaatggcaaggtcagtgcaaaagagagcatctagttactgaggtactgaggtcgatccatagaaagatgtctattacagatgtctccgtagttcataatgaattgatgatttggtagtaagcttcacaatactgttctgaccagctcttattggtttccatttttgtcacataattttatacagttgttaacactgcatgtttcctctaatcttatgatgttgctccaaaaactaatttttcaaattttggcaggctgagagaccaacattatcaaacgtcaatctggatgtacctatgggcagtttggttgcaatagtaggaagcactggggaggggaagacttctcttatttctgcaatgcttggtgaaataccaccagtgtcaggatcgaatacatcggtggtccttcgtggaacagtggcttatgttcctcaagtttcatggatcttcaatgcaactgtgagacacaatatctccatttatgttgattgttatattgaaccttttatggcttctacagatataatgttatcatctctgcaggtccgggacaatatattgtttgggtctcccttccaacctccacgctacgagaaagcaatagatgtcacttcattacggcatgaccttgacttactcccagtaagttctaactatttcagtcttcagagatcgttctggaaatttttgacactcgagctttcatttgatagataacttgctggggagtgtgttttttttttcatttgtcatgttgaaattatgttgccaatacctttttctatcttggttttgcgctttatattcttgtacctgcaaattgacaagaaaatatatcttcttatgcttgttgtcagggtggtgacctaacagagattggagaaagaggagttaacattagtgggggacaaaaacagagagtctcaatggcaagagctgtttattctgattcagatgtctacatatttgatgatccactgagtgcattagatgcccatgttggtcgacaggtgcattcatcttgctaccactgcggttcatgctttcaattttacagtctcccccgcattatcaaatctgccagtggcagttggtaatggtgtcaatgttttgtggatatcagcatcttttttgtcgaaattcctttttactgagattcccttgtgtctgttttacttccctgggacatgaaaacatgaggttctatgccattggtttggcatgtgtcagccatgtttctgttatgaacatcacattgctatgcatttttttcttatgcccgtttactgcagataggcacactttctggttatagagtaattgcgtcgataagatatggtacaccatttgtcacggtagatacttaaatctgtttgttgtgaccgaactaatgttgtgaataattatatgccaaatcatcctatatgctgtgctttggtagcctgctacaagtgatgtaccttggaggctgtttggatccctcttaccaaaactagattttgttaaacagattttagatcctgacaaattgttatgtgatccaaacaaagattttcttctgtatgaaaatatttcagttcaactaaataatcttaattactgatgccctattatcacatgcttatgcaggtatttgacaaatgtattaaagaagagctacagcacaaaactagggttcttgttaccaatcagctgcattttctaccatatgtggataaaatactggtagtccatgatggtgtaattaaagaagaaggtacctttgatgaacttagcaactctggggaactctttaagaagctcatggaaaatgctggaaagatggaagaacagatggaagagaaacaggacgaaagcaaaagacaggatgacataaaacaccctgaaaatgggggcagtgtaatagctgatggtgatatgcagaagagtcaggacacttctaataaaacaaaacaggggaaatctgttctgattaaacaagaagaaagggaaactggagttataagtgcaaaggtcctttcacggtaaaagattgttgtctcattgtatggtagtatatttcatgtataatacagatagaagaaaggaaacaggagagggagggaggcataatattaaacatatttatagatcatgatgacctggaatatctgcttacttaatacctttacctttgcagctacaaaaatgcattgggagggatctgggtggtatctgttctctttttttgctatgcactcactgaagttcttcgcatttcaagtagcacatggttgagcgtttggacagatcaaggttctacgaaaatccatggccctggttactacaatttaatctacggccttctttcttttgggcaggtacttgtaccatgattttcatcttcttttcttgattagaatttgaatttatccaattgatcacatgctggctgcaaaatatgacattgtgttgtttataggttctagtcactctaacgaattcttattggctgatcacatcaagtcttcgagcagccaagaggttgcatgatgccatgctccggtctatattaagagctcccatggtattttttcacaccaatccacttggacgaatcatcaacagattttcaaaggatttgggtgacattgacaggaatgtagccatctttgtgaatatgtttatggcacaaatatctcagttgctctcaacatttgttctcattggcattgttagcactatgtctctgtgggctatcatgccacttctgattttattttatgcagcttacctctattaccaggtacttttgtgacagctgacttttttaaaaaatatatattaaagattgtattgttagtcctaacttcttgaatcttgacatctcatacctgtcttttcccagactacgtctcgtgaggtaaagcgcctggattccattacaaggtctcctgtgtatgctcaattttcagaggcattgaatggtctgtctacaatccgtgcctacaaagcctatgatagaatggcaaacatcaatgggaaatcaatggacaacaacatcagattcactcttgtgaacatgagttcaaacagatggctagccatccgtctggaaacattgggtggcatcatgatatggttcacagcaacatttgctgtcatgcaaaaccaacgagcagaaaatcaaaaggcttttgcctccacaatgggtcttcttcttacttacaccctcaatatcaccaatttgctcacggctgttcttcgtcttgctagtctggctgaaaacagtttaaatgctgttgagcgggtaggaacatacattgagttgccttctgaggctcctcctgtcattgaggatagcaggccacctcctggttggccatcatctggtgttgtcaagtttgaagacgtcgtgcttcggtaccgaccagaacttcctcctgttcttcatggcatatctttcatcattaatggcagcgaaaaggtaggaatagttggcagaacaggagctggtaaatctagcatgctgaatgctctgttccgtattgtggaactggaacgagggagaatattggttgatgattgtgacacttctaaatttggaatttgggacctacgaaaagtactaggaataataccacaggcacctgtcctattttcaggtttgtttgtgaaaatacatgatatatcttctcgtttgcttttcttacatcataaatgttttttttttttggtactattgtccaggttctgttcgatttaatctggatccttttaatgagcacaatgatgcggatctctgggaagcgcttgaaagggctcatctaaaagatgtcataaggaggaatgctctgggactagatgctgaggtacgtagtttgtttatcctttatttagttcagaccattatttcacttttttttaagacgatgtatgcctgtatagacttgtcagttttttttatgtcagtctatagtttagcagtttaccttttggtattcaatatattccaagagtttgctttcggaataaaacaggattgttatagaattatgctactgctgctttctccttgtagttaatacacaatattgaataaactttctttatagtagttaccttgttttttttaaacaaacacttcggaaaatttctgtggaacttcatcttgttaacacactcaggtctctgaggctggtgaaaattttagcgttggacagcggcagctactgagtttagctcgtgcattgctgcggagggcaaagatacttgttcttgatgaggcaacagcagctgttgatgttcgaacggatgctcttatacagaagacaatcagggaagaattcaagagttgtacaatgcttataattgctcaccgcctgaacactgttattgactgtgacaggttgcttattctaagtgctgggaaggtatgcttttgaagagatgtttcttcttttgttctggtgctttgcctctcatttgaatagtacaacaataaattcctgataatatcaacatgcttgataggtgtaatttccaaagtaaccaatgaaggattgttgcttgtctgttttggactcttgatttgaatgttgctttccttagctcctggagcttggtttccacaattttccaactgaactaaaacgtggtcatagtgagcttgtctgtaattagtatcatactatcctttacaatctctatattataaagtgttatttcaactgtcatgtttccagtgttctgttttcatattttattagtattatagatgacttgtaagagcattgaattcaccctgaaaagactcttggtatttattaacctagtgagatagatggcttgaaataattctgttcactctgacaaatagagagctaaacacgttcaggttgcactcttttcgtgctcagcctattatttttcttgattgcttaatttttggcacctcagttatgaaactactacttttcaggttttggagttcgactctcctgagaatcttttgagcaatgagcacagtgctttctccaagatggttcagagtacaggacctagcaatgcagagtacctgaaggtgtttttctatcatctcgatgttcttgaaatttgtgtactacagcataaaacagcgtttgataaataataatgagtgatgctgttcaatccatgttacactccatcgctcaagttttcacagttggctttcagcgaaattattgatatgtgttctgattcaacttttttttgactgaattattgacagactcttgtatttggggatggtgaggagaggttgcgaaaggaagaaagcaagatgcaagatatccagaggaagtgggtagcatctaaccgatgggctgttgctgcgcagtttgcgcttgccgctagccttgcctcttcacacagtgaccttcttgcactggaggctgctgagggaaacaacatcctcaggaaaacaaaggatgcagtaatcactctacagaatgtcctcgaagggaagcacaacaccgaaattgatgatacactcgctcagtatgaggttccatctgataggtggtggtcatcgctatacaaagtcatggaaggtacgttgatcacttcattttaacacctttctcttgtgcgatgcttcagagtagcatgtattgtgtgcaatataagtttattacactagtcccagtgtaaactggctaatattggtccgtcagagattctgatgaaaccaatagttaacagtgagtaccggattggtgcttggtcgatgcaaaagtccatatgttaacatgaagagaactgctgaaatataacgatagggataagattttcttggaccaacaaacatactaacacttgacaaaagcatgtcctccacatgccattgtcatgatctctttggtattcaaatctaaccctatcaatctgaccttttgatcaaatatgtatatctattttttttcaggacttgccatgatgagccgattgggtcgcaaccgtctacagcaaccaagttataattttgaaaacaacagttctattgactgggaccaaatgtagaagcttgtgacggcaaagtaaggtgaccagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattaaactatcagtgtttgacaggatatattggcgggtaaac

SEQ ID NO: 35 - DNA, 11042 bp, synthetic construct
  source 1..11042: mol_type = other DNA
atgggttttgatccactggaatggtactgccagccggtcaatggcggggtgtggtcagatgtagagaatgcttttggtgcatacacaccatgtggcactgaaaccttggtggtgtgcatctcatactttgcactcttcggtgtttgcttctatcggatatggaggacgacgagggactacacggtgcagcgctacaaattgcggtcgccatactacaactatatgctcggcctgcttgtggtgttgtgcatagctgaatcactgtatcggatcgctactggcacctccataatgaacttggatggggagactagccttgctccatttgaggtgagaacatgtcccaagtataagtgtatcagcacgtagagttaagcacacttgttcagaattttacttctaccgaattcttgcatcccttgaccttttcaatcacgtaattctgcctctacaggttacctcatctatcattgagattgctgcttggtgttgtatgcttgtaatgattgcacttgagacaagaatttacatctatgagtttagatggtacatccggtttgtggtcatttacatcttggttggggaagctgctatgttcaatcttgtgctttcagtaaggcagtattatagttcgaggtatagtacacatgagacatgctttattcatgaaatttattgaatgttgattgatagagaactttatactctaaatttttcaatgaagactgacttctgtattttggttatgcagttcaatattttacttgtactgcagtgagataatattcaaggtttgcattcttgttatcttaataatcgtactgtagtgatatttgtcgtatactgaacttatcatctctgcagctcttgtttggaattctcatggtggtttatctgcctagcttggattcctatccgggttatactccagtcaggcatgaggcacttgttgataatactgattacgaacctctgcctggtggggagcagatttgccctgagaggcatgctaatatattttccagtgagtaaatataataagttgtgcggttgtgccacactattgcatacatccataatgttttgtttgtcctcataatttctctaaatttctagaagcacactgaggctttttggttgtcttaccttgctagggatattcttttcatggatgactcctctaatgcaacaaggatttaaaaggcccatcaccgataaggatatttggaaattagacagttgggatgagactgaaacattgtacaaccggtatgttctgcaaccatttttactgaaatgagaaacacttgtatatgcagatcttaaccttataatctttcctcaggttccaaaaatgctggaacaatgagcttcaaaaaccaaagccttggctgttacgggctctgcatagcagccttggtggaaggtatatttgtacagatgatagctatattttccagtaaacctttagggtggtcccttcttgctacattgtttgtgttcacgtagtgattggtttcattcattttcaggttttggctaggaggattttttaaggtaggtaaatgtatctcgacttttgtaaagttatccttcaatttcgtttgcatttttgtttgatggcaagcattttgtgttattcctggcaactcatcctttttcctccaaaattttgtagattggcaatgatgcttctcagtttgttggcccactcatattgaacctcttgttggaggtatgcttctagtttcatgaaccaatggcaataaaattgcattgcattacctttacaagccttattctactattgtttttaccatcaacaaacttgtgaagtaattcaaaacatactgggtaattgtgcagattttaatatttatatatgaaagaatagatccaacttttctcttatgttgttgagatgctagtgttaggtgctttgtaccactaaaggtcaatgttcttttggcagtctatgcaaaaaggtgatccatcttggagcggttacatctatgctttctcaatcttcgctggagtggtatggattattttcttctattgcatgtttcacacttatatgtcttgttgattcagtgtatttgaacttccaagcaacaattatgctgcatgtgagccaaaactggacagacttttgatcacttaccttttgtcgaccattctgcagtcactgggtgttctttctgaagcacagtactttcagaatgtcatgcgaacgggtttcaggttgaggtcaacattggtaggtgttgcttgtttagcatggtttaatatgatgtcaacaggcaaccagcaaaccacttgttccactatttttctggaccactcttttttttttctctactcccatggttatcttgagcattgaacttcctgatttatgcatcaaggacatgtgtaattgtaatattaagtttagacctttatttttcttatgttcaccatgatataatattatctggtaataattgactttcctgcaagtaacactaccatagccatctcagctgctgttatatgtttagttacacgcttatgccatgattctgttgtttcgcagatcgcagctgttttccgtaagtctttgcgactaactaatgatagtcgaaagaagtttgcctctgggaggataaccaatttgatttcaactgacgcagaatctcttcaggtgtgattttatttcataggtttaatcagtgttacccagttcttcaacattattgattttaaatattgtttggcaacataaaattagataaaaaaccaaatgatatgttagataccttgttgatgtctgaattgataagtaaatggaaaaccgtggaaatgttttgttaggaaagagcaatggagcaagaaatacaatatgtgctacaatatggagctatggttatttatattttatgttgtacatttggtttgtactttttttacttttgcagcaagtgtgccagcaacttcacagtctatggtctgctcctttccgcattgttattgccatggtccttctatacgcacaactaggccctgctgcattggtcggtgcagccatgttggttcttttgttcccaattcaggtagatttctctttagacattcaattatggaaggtcttatgttgcatttttaactttctatctggtccctacattaccaatatctactttgtgttttttatattttgattctttttagggaatgtgcagaaagattgtactgatatatcgattcttatatggagcctagagttcacattagagtctttcatgccatcatttagttgttgattctacttcctgttatctatcctgaggttgtattgtagtactgtttgtacgtctgcttagttttctgccataccccgtaagaaattctttgatgttgtccataaattggagttgatcaagtcaatgcttaaatccctaatatttggctaatccatattgctcatgctttctcttttgaagcttataatgcaaattgccagggcacttaacagttgctgattttcagattatgctatttttatgaatgatatatgcatttcaaataacgattttacttttaagcaaacttttgctactttcaacatagatctgactgttaacattttctttatttaatttaccagactgttatcataagcaaaatgcaaaaacttaccaaggaagggttgcaaaggactgacaggagaatcagtctaatgaatgaaatattagctgcaatggatacggtcaagtatgctttacatttacttgtgtttttactagaagtagtactaaaattttggtataaactgttccaaacattttcttggttagatgttatgcttgggagcaaagtttccagtcaaaagtgcaggacatccgtgatgatgaaatttcatggttccgcagcgctcaattgcttgctgcggtatgtccattttgtatttaagttatctttcacttgtgtcaatttcattctgctatatatgatctcttttttaatcgtgcatccatgaactctccttattacacaataagttttggagttgcatgctatagtatcgattgtactggtgcttcctggtcctataaagtttgtttacaataatgtacttagttactcaaacttaaaatagatatgagaatgagttgttgcatcccttattgtcggctcactattatcttttgtatttttccttgtctgctgcagctgaatagcttcatcctaaacagtatccccgttattgtcactgtggtttcatttggtgtatactctctgctgggaggtgacttgacaccagcaaaggcgttcacatcactttcactgtttgctgtcttaagatttccactttttatgctgccaaatctgataactcaggtgtgcaaaaagctgtaaattatgtcttgttaagtttcccactttctgtacaagttaagaagcatatcattttctgaacttatatgtttggattgataatcattcttgtaccagttaattttcatctgtctttggcagagaaaattagtacaatggtttttacaattttggtcacagtccaatgctggcttgagatagttctctgcttttcttgttcaacgactcttttgttagtactcatagtcaccttactattttttttccttttaaatttgacaggtggttaattgtaaagtgtcattgaaacgtctggaagatctcctcttggctgaagagagattacttctgcccaatccacctcttgatcctgagcttccagcaatttctattaagaatggatatttttcatgggaatcgcaggtaatttcatgtttcagatatttgcctggtatctaaatatatctatgttgactatcacacattcaactctcatcattcattgatattataatataacacatatattattgttctgttgtctggagtactgaaaagaaacaaaagtttgggcaaataaatactgaattagagtattgatagactagcatgcttcatgaatggcaaggtcagtgcaaaagagagcatctagttactgaggtactgaggtcgatccatagaaagatgtctattacagatgtctccgtagttcataatgaattgatgatttggtagtaagcttcacaatactgttctgaccagctcttattggtttccatttttgtcacataattttatacagttgttaacactgcatgtttcctctaatcttatgatgttgctccaaaaactaatttttcaaattttggcaggctgagagaccaacattatcaaacgtcaatctggatgtacctatgggcagtttggttgcaatagtaggaagcactggggaggggaagacttctcttatttctgcaatgcttggtgaaataccaccagtgtcaggatcgaatacatcggtggtccttcgtggaacagtggcttatgttcctcaagtttcatggatcttcaatgcaactgtgagacacaatatctccatttatgttgattgttatattgaaccttttatggcttctacagatataatgttatcatctctgcaggtccgggacaatatattgtttgggtctcccttccaacctccacgctacgagaaagcaatagatgtcacttcattacggcatgaccttgacttactcccagtaagttctaactatttcagtcttcagagatcgttctggaaatttttgacactcgagctttcatttgatagataacttgctggggagtgtgttttttttttcatttgtcatgttgaaattatgttgccaatacctttttctatcttggttttgcgctttatattcttgtacctgcaaattgacaagaaaatatatcttcttatgcttgttgtcagggtggtgacctaacagagattggagaaagaggagttaacattagtgggggacaaaaacagagagtctcaatggcaagagctgtttattctgattcagatgtctacatatttgatgatccactgagtgcattagatgcccatgttggtcgacaggtgcattcatcttgctaccactgcggttcatgctttcaattttacagtctcccccgcattatcaaatctgccagtggcagttggtaatggtgtcaatgttttgtggatatcagcatcttttttgtcgaaattcctttttactgagattcccttgtgtctgttttacttccctgggacatgaaaacatgaggttctatgccattggtttggcatgtgtcagccatgtttctgttatgaacatcacattgctatgcatttttttcttatgcccgtttactgcagataggcacactttctggttatagagtaattgcgtcgataagatatggtacaccatttgtcacggtagatacttaaatctgtttgttgtgaccgaactaatgttgtgaataattatatgccaaatcatcctatatgctgtgctttggtagcctgctacaagtgatgtaccttggaggctgtttggatccctcttaccaaaactagattttgttaaacagattttagatcctgacaaattgttatgtgatccaaacaaagattttcttctgtatgaaaatatttcagttcaactaaataatcttaattactgatgccctattatcacatgcttatgcaggtatttgacaaatgtattaaagaagagctacagcacaaaactagggttcttgttaccaatcagctgcattttctaccatatgtggataaaatactggtagtccatgatggtgtaattaaagaagaaggtacctttgatgaacttagcaactctggggaactctttaagaagctcatggaaaatgctggaaagatggaagaacagatggaagagaaacaggacgaaagcaaaagacaggatgacataaaacaccctgaaaatgggggcagtgtaatagctgatggtgatatgcagaagagtcaggacacttctaataaaacaaaacaggggaaatctgttctgattaaacaagaagaaagggaaactggagttataagtgcaaaggtcctttcacggtaaaagattgttgtctcattgtatggtagtatatttcatgtataatacagatagaagaaaggaaacaggagagggagggaggcataatattaaacatatttatagatcatgatgacctggaatatctgcttacttaatacctttacctttgcagctacaaaaatgcattgggagggatctgggtggtatctgttctctttttttgctatgcactcactgaagttcttcgcatttcaagtagcacatggttgagcgtttggacagatcaaggttctacgaaaatccatggccctggttactacaatttaatctacggccttctttcttttgggcaggtacttgtaccatgattttcatcttcttttcttgattagaatttgaatttatccaattgatcacatgctggctgcaaaatatgacattgtgttgtttataggttctagtcactctaacgaattcttattggctgatcacatcaagtcttcgagcagccaagaggttgcatgatgccatgctccggtctatattaagagctcccatggtattttttcacaccaatccacttggacgaatcatcaacagattttcaaaggatttgggtgacattgacaggaatgtagccatctttgtgaatatgtttatggcacaaatatctcagttgctctcaacatttgttctcattggcattgttagcactatgtctctgtgggctatcatgccacttctgattttattttatgcagcttacctctattaccaggtacttttgtgacagctgacttttttaaaaaatatatattaaagattgtattgttagtcctaacttcttgaatcttgacatctcatacctgtcttttcccagactacgtctcgtgaggtaaagcgcctggattccattacaaggtctcctgtgtatgctcaattttcagaggcattgaatggtctgtctacaatccgtgcctacaaagcctatgatagaatggcaaacatcaatgggaaatcaatggacaacaacatcagattcactcttgtgaacatgagttcaaacagatggctagccatccgtctggaaacattgggtggcatcatgatatggttcacagcaacatttgctgtcatgcaaaaccaacgagcagaaaatcaaaaggcttttgcctccacaatgggtcttcttcttacttacaccctcaatatcaccaatttgctcacggctgttcttcgtcttgctagtctggctgaaaacagtttaaatgctgttgagcgggtaggaacatacattgagttgccttctgaggctcctcctgtcattgaggatagcaggccacctcctggttggccatcatctggtgttgtcaagtttgaagacgtcgtgcttcggtaccgaccagaacttcctcctgttcttcatggcatatctttcatcattaatggcagcgaaaaggtaggaatagttggcagaacaggagctggtaaatctagcatgctgaatgctctgttccgtattgtggaactggaacgagggagaatattggttgatgattgtgacacttctaaatttggaatttgggacctacgaaaagtactaggaataataccacaggcacctgtcctattttcaggtttgtttgtgaaaatacatgatatatcttctcgtttgcttttcttacatcataaatgttttttttttttggtactattgtccaggttctgttcgatttaatctggatccttttaatgagcacaatgatgcggatctctgggaagcgcttgaaagggctcatctaaaagatgtcataaggaggaatgctctgggactagatgctgaggtacgtagtttgtttatcctttatttagttcagaccattatttcacttttttttaagacgatgtatgcctgtatagacttgtcagttttttttatgtcagtctatagtttagcagtttaccttttggtattcaatatattccaagagtttgctttcggaataaaacaggattgttatagaattatgctactgctgctttctccttgtagttaatacacaatattgaataaactttctttatagtagttaccttgttttttttaaacaaacacttcggaaaatttctgtggaacttcatcttgttaacacactcaggtctctgaggctggtgaaaattttagcgttggacagcggcagctactgagtttagctcgtgcattgctgcggagggcaaagatacttgttcttgatgaggcaacagcagctgttgatgttcgaacggatgctcttatacagaagacaatcagggaagaattcaagagttgtacaatgcttataattgctcaccgcctgaacactgttattgactgtgacaggttgcttattctaagtgctgggaaggtatgcttttgaagagatgtttcttcttttgttctggtgctttgcctctcatttgaatagtacaacaataaattcctgataatatcaacatgcttgataggtgtaatttccaaagtaaccaatgaaggattgttgcttgtctgttttggactcttgatttgaatgttgctttccttagctcctggagcttggtttccacaattttccaactgaactaaaacgtggtcatagtgagcttgtctgtaattagtatcatactatcctttacaatctctatattataaagtgttatttcaactgtcatgtttccagtgttctgttttcatattttattagtattatagatgacttgtaagagcattgaattcaccctgaaaagactcttggtatttattaacctagtgagatagatggcttgaaataattctgttcactctgacaaatagagagctaaacacgttcaggttgcactcttttcgtgctcagcctattatttttcttgattgcttaatttttggcacctcagttatgaaactactacttttcaggttttggagttcgactctcctgagaatcttttgagcaatgagcacagtgctttctccaagatggttcagagtacaggacctagcaatgcagagtacctgaaggtgtttttctatcatctcgatgttcttgaaatttgtgtactacagcataaaacagcgtttgataaataataatgagtgatgctgttcaatccatgttacactccatcgctcaagttttcacagttggctttcagcgaaattattgatatgtgttctgattcaacttttttttgactgaattattgacagactcttgtatttggggatggtgaggagaggttgcgaaaggaagaaagcaagatgcaagatatccagaggaagtgggtagcatctaaccgatgggctgttgctgcgcagtttgcgcttgccgctagccttgcctcttcacacagtgaccttcttgcactggaggctgctgagggaaacaacatcctcaggaaaacaaaggatgcagtaatcactctacagaatgtcctcgaagggaagcacaacaccgaaattgatgatacactcgctcagtatgaggttccatctgataggtggtggtcatcgctatacaaagtcatggaaggtacgttgatcacttcattttaacacctttctcttgtgcgatgcttcagagtagcatgtattgtgtgcaatataagtttattacactagtcccagtgtaaactggctaatattggtccgtcagagattctgatgaaaccaatagttaacagtgagtaccggattggtgcttggtcgatgcaaaagtccatatgttaacatgaagagaactgctgaaatataacgatagggataagattttcttggaccaacaaacatactaacacttgacaaaagcatgtcctccacatgccattgtcatgatctctttggtattcaaatctaaccctatcaatctgaccttttgatcaaatatgtatatctattttttttcaggacttgccatgatgagccgattgggtcgcaaccgtctacagcaaccaagttataattttgaaaacaacagttctattgactgggaccaaatgtag

SEQ ID NO: 36 - DNA, 4887 bp, synthetic construct
  source 1..4887: mol_type = other DNA
atgggttttgatccactggaatggtactgccagccggtcaatggcggggtgtggtcagatgtagagaatgcttttggtgcatacacaccatgtggcactgaaaccttggtggtgtgcatctcatactttgcactcttcggtgtttgcttctatcggatatggaggacgacgagggactacacggtgcagcgctacaaattgcggtcgccatactacaactatatgctcggcctgcttgtggtgttgtgcatagctgaatcactgtatcggatcgctactggcacctccataatgaacttggatggggagactagccttgctccatttgaggttacctcatctatcattgagattgctgcttggtgttgtatgcttgtaatgattgcacttgagacaagaatttacatctatgagtttagatggtacatccggtttgtggtcatttacatcttggttggggaagctgctatgttcaatcttgtgctttcagtaaggcagtattatagttcgagttcaatattttacttgtactgcagtgagataatattcaagctcttgtttggaattctcatggtggtttatctgcctagcttggattcctatccgggttatactccagtcaggcatgaggcacttgttgataatactgattacgaacctctgcctggtggggagcagatttgccctgagaggcatgctaatatattttccaggatattcttttcatggatgactcctctaatgcaacaaggatttaaaaggcccatcaccgataaggatatttggaaattagacagttgggatgagactgaaacattgtacaaccggttccaaaaatgctggaacaatgagcttcaaaaaccaaagccttggctgttacgggctctgcatagcagccttggtggaaggttttggctaggaggattttttaagattggcaatgatgcttctcagtttgttggcccactcatattgaacctcttgttggagtctatgcaaaaaggtgatccatcttggagcggttacatctatgctttctcaatcttcgctggagtgtcactgggtgttctttctgaagcacagtactttcagaatgtcatgcgaacgggtttcaggttgaggtcaacattgatcgcagctgttttccgtaagtctttgcgactaactaatgatagtcgaaagaagtttgcctctgggaggataaccaatttgatttcaactgacgcagaatctcttcagcaagtgtgccagcaacttcacagtctatggtctgctcctttccgcattgttattgccatggtccttctatacgcacaactaggccctgctgcattggtcggtgcagccatgttggttcttttgttcccaattcagactgttatcataagcaaaatgcaaaaacttaccaaggaagggttgcaaaggactgacaggagaatcagtctaatgaatgaaatattagctgcaatggatacggtcaaatgttatgcttgggagcaaagtttccagtcaaaagtgcaggacatccgtgatgatgaaatttcatggttccgcagcgctcaattgcttgctgcgctgaatagcttcatcctaaacagtatccccgttattgtcactgtggtttcatttggtgtatactctctgctgggaggtgacttgacaccagcaaaggcgttcacatcactttcactgtttgctgtcttaagatttccactttttatgctgccaaatctgataactcaggtggttaattgtaaagtgtcattgaaacgtctggaagatctcctcttggctgaagagagattacttctgcccaatccacctcttgatcctgagcttccagcaatttctattaagaatggatatttttcatgggaatcgcaggctgagagaccaacattatcaaacgtcaatctggatgtacctatgggcagtttggttgcaatagtaggaagcactggggaggggaagacttctcttatttctgcaatgcttggtgaaataccaccagtgtcaggatcgaatacatcggtggtccttcgtggaacagtggcttatgttcctcaagtttcatggatcttcaatgcaactgtccgggacaatatattgtttgggtctcccttccaacctccacgctacgagaaagcaatagatgtcacttcattacggcatgaccttgacttactcccaggtggtgacctaacagagattggagaaagaggagttaacattagtgggggacaaaaacagagagtctcaatggcaagagctgtttattctgattcagatgtctacatatttgatgatccactgagtgcattagatgcccatgttggtcgacaggtatttgacaaatgtattaaagaagagctacagcacaaaactagggttcttgttaccaatcagctgcattttctaccatatgtggataaaatactggtagtccatgatggtgtaattaaagaagaaggtacctttgatgaacttagcaactctggggaactctttaagaagctcatggaaaatgctggaaagatggaagaacagatggaagagaaacaggacgaaagcaaaagacaggatgacataaaacaccctgaaaatgggggcagtgtaatagctgatggtgatatgcagaagagtcaggacacttctaataaaacaaaacaggggaaatctgttctgattaaacaagaagaaagggaaactggagttataagtgcaaaggtcctttcacgctacaaaaatgcattgggagggatctgggtggtatctgttctctttttttgctatgcactcactgaagttcttcgcatttcaagtagcacatggttgagcgtttggacagatcaaggttctacgaaaatccatggccctggttactacaatttaatctacggccttctttcttttgggcaggttctagtcactctaacgaattcttattggctgatcacatcaagtcttcgagcagccaagaggttgcatgatgccatgctccggtctatattaagagctcccatggtattttttcacaccaatccacttggacgaatcatcaacagattttcaaaggatttgggtgacattgacaggaatgtagccatctttgtgaatatgtttatggcacaaatatctcagttgctctcaacatttgttctcattggcattgttagcactatgtctctgtgggctatcatgccacttctgattttattttatgcagcttacctctattaccagactacgtctcgtgaggtaaagcgcctggattccattacaaggtctcctgtgtatgctcaattttcagaggcattgaatggtctgtctacaatccgtgcctacaaagcctatgatagaatggcaaacatcaatgggaaatcaatggacaacaacatcagattcactcttgtgaacatgagttcaaacagatggctagccatccgtctggaaacattgggtggcatcatgatatggttcacagcaacatttgctgtcatgcaaaaccaacgagcagaaaatcaaaaggcttttgcctccacaatgggtcttcttcttacttacaccctcaatatcaccaatttgctcacggctgttcttcgtcttgctagtctggctgaaaacagtttaaatgctgttgagcgggtaggaacatacattgagttgccttctgaggctcctcctgtcattgaggatagcaggccacctcctggttggccatcatctggtgttgtcaagtttgaagacgtcgtgcttcggtaccgaccagaacttcctcctgttcttcatggcatatctttcatcattaatggcagcgaaaaggtaggaatagttggcagaacaggagctggtaaatctagcatgctgaatgctctgttccgtattgtggaactggaacgagggagaatattggttgatgattgtgacacttctaaatttggaatttgggacctacgaaaagtactaggaataataccacaggcacctgtcctattttcaggttctgttcgatttaatctggatccttttaatgagcacaatgatgcggatctctgggaagcgcttgaaagggctcatctaaaagatgtcataaggaggaatgctctgggactagatgctgaggtctctgaggctggtgaaaattttagcgttggacagcggcagctactgagtttagctcgtgcattgctgcggagggcaaagatacttgttcttgatgaggcaacagcagctgttgatgttcgaacggatgctcttatacagaagacaatcagggaagaattcaagagttgtacaatgcttataattgctcaccgcctgaacactgttattgactgtgacaggttgcttattctaagtgctgggaaggttttggagttcgactctcctgagaatcttttgagcaatgagcacagtgctttctccaagatggttcagagtacaggacctagcaatgcagagtacctgaagactcttgtatttggggatggtgaggagaggttgcgaaaggaagaaagcaagatgcaagatatccagaggaagtgggtagcatctaaccgatgggctgttgctgcgcagtttgcgcttgccgctagccttgcctcttcacacagtgaccttcttgcactggaggctgctgagggaaacaacatcctcaggaaaacaaaggatgcagtaatcactctacagaatgtcctcgaagggaagcacaacaccgaaattgatgatacactcgctcagtatgaggttccatctgataggtggtggtcatcgctatacaaagtcatggaaggacttgccatgatgagccgattgggtcgcaaccgtctacagcaaccaagttataattttgaaaacaacagttctattgactgggaccaaatgtag

SEQ ID NO: 37 - protein, 1628 aa, synthetic construct
MGFDPLEWYCQPVNGGVWSDVENAFGAYTPCGTETLVVCISYFALFGVCFYRIWRTTRDYTVQRYKLRSPYYNYMLGLLVVLCIAESLYRIATGTSIMNLDGETSLAPFEVTSSIIEIAAWCCMLVMIALETRIYIYEFRWYIRFVVIYILVGEAAMFNLVLSVRQYYSSSSIFYLYCSEIIFKLLFGILMVVYLPSLDSYPGYTPVRHEALVDNTDYEPLPGGEQICPERHANIFSRIFFSWMTPLMQQGFKRPITDKDIWKLDSWDETETLYNRFQKCWNNELQKPKPWLLRALHSSLGGRFWLGGFFKIGNDASQFVGPLILNLLLESMQKGDPSWSGYIYAFSIFAGVSLGVLSEAQYFQNVMRTGFRLRSTLIAAVFRKSLRLTNDSRKKFASGRITNLISTDAESLQQVCQQLHSLWSAPFRIVIAMVLLYAQLGPAALVGAAMLVLLFPIQTVIISKMQKLTKEGLQRTDRRISLMNEILAAMDTVKCYAWEQSFQSKVQDIRDDEISWFRSAQLLAALNSFILNSIPVIVTVVSFGVYSLLGGDLTPAKAFTSLSLFAVLRFPLFMLPNLITQVVNCKVSLKRLEDLLLAEERLLLPNPPLDPELPAISIKNGYFSWESQAERPTLSNVNLDVPMGSLVAIVGSTGEGKTSLISAMLGEIPPVSGSNTSVVLRGTVAYVPQVSWIFNATVRDNILFGSPFQPPRYEKAIDVTSLRHDLDLLPGGDLTEIGERGVNISGGQKQRVSMARAVYSDSDVYIFDDPLSALDAHVGRQVFDKCIKEELQHKTRVLVTNQLHFLPYVDKILVVHDGVIKEEGTFDELSNSGELFKKLMENAGKMEEQMEEKQDESKRQDDIKHPENGGSVIADGDMQKSQDTSNKTKQGKSVLIKQEERETGVISAKVLSRYKNALGGIWVVSVLFFCYALTEVLRISSSTWLSVWTDQGSTKIHGPGYYNLIYGLLSFGQVLVTLTNSYWLITSSLRAAKRLHDAMLRSILRAPMVFFHTNPLGRIINRFSKDLGDIDRNVAIFVNMFMAQISQLLSTFVLIGIVSTMSLWAIMPLLILFYAAYLYYQTTSREVKRLDSITRSPVYAQFSEALNGLSTIRAYKAYDRMANINGKSMDNNIRFTLVNMSSNRWLAIRLETLGGIMIWFTATFAVMQNQRAENQKAFASTMGLLLTYTLNITNLLTAVLRLASLAENSLNAVERVGTYIELPSEAPPVIEDSRPPPGWPSSGVVKFEDVVLRYRPELPPVLHGISFIINGSEKVGIVGRTGAGKSSMLNALFRIVELERGRILVDDCDTSKFGIWDLRKVLGIIPQAPVLFSGSVRFNLDPFNEHNDADLWEALERAHLKDVIRRNALGLDAEVSEAGENFSVGQRQLLSLARALLRRAKILVLDEATAAVDVRTDALIQKTIREEFKSCTMLIIAHRLNTVIDCDRLLILSAGKVLEFDSPENLLSNEHSAFSKMVQSTGPSNAEYLKTLVFGDGEERLRKEESKMQDIQRKWVASNRWAVAAQFALAASLASSHSDLLALEAAEGNNILRKTKDAVITLQNVLEGKHNTEIDDTLAQYEVPSDRWWSSLYKVMEGLAMMSRLGRNRLQQPSYNFENNSSIDWDQM

SEQ ID NO: 38 - protein, 519 aa, synthetic construct
MASKPSSRAESNQAAAAVPSLYGRALPSPPAVEFASAEGRRLFAEALQGGTMQGFFSLVSVFQTQSEPAFCGLATLAVVLNALRIDPGRRWKGPWRWFDESMLDCCEHLDTVRAEGITFGKVACLAHCSGADVRTFRAAQATLADLRRHLLRCASSQDCHLVASYHRKLLGQTGTGHFSPIGGYHAGQDMALILDVARFKYPPHWIPLPLLWEAMNTIDEATGLLRGFMLISRNTEAPLLIRAVNCRDESWQSMAKYCIEVVPNLLRDNSVDNVLTILSRLVNHLPPNAGNFIKWVIEVRRQEEGGSSPSKEANEMPFLKEKVLQQIRDTKLFQLVHKLQCSKQPCCSCSSLTDEDSISQIAASVCCEATALLSGNLSSRDGLFFSETCSGCTQVNDEGLKNVITGKVVSEGNGHVDKLSPISSTETCFCNSTLSNETVNYPSNTDILTVLLLSLHPSTWLCIEDEKLKAEFQSLVSTDDLPDPLKLEILHLRRQLRYLKACREKEAYEDTWPQPWEQC

SEQ ID NO: 39 - protein, 1004 aa, synthetic construct
MAGKDEAEGLEARLLLLPPEAAAEEPTRCGGGDGGGGGRKRKKTYLDVLGVCCSAEVALVERLLAPLDGVRVVSVVVASRTVVVEHDPAAAPESAIVKALNKAGLEASVRAYGSSGVVSRWPSPYIVASGVLLTASFFEWLFPPLQCLAVAAVVAGAPPMVRRGFAAASRLSLDINVLMLIAVAGALCLGDYTEAGAIVFLFTTAEWLETLACTKASAGMSSLMGMLPVKAVIATTGEVVSVRDVRVGDVVAVRAGEIVPVDGVVVDGQSEVDERSLTGESFPVPKQPHSEVWAGTMNFDGYIAVRTTALAENSTVAKMERLVEAAQNSRSKTQRLIDSCAKYYTPAVVVVAAGVALIPALLGADGLEQWWKLALVMLVSACPCALVLSTPVASFCAMLRAARMGIFIKGGDVLESLGEIRAVAFDKTGTITRGEFSIDSFHLVGDHKVEMDHLLYWIASIESKSSHPMAAALVEYAQSKSIQPNPENVGDFRIYPGEGIYGEIHGKHIYIGNRRTLARASSPQSTQEMGEMIKGVSIGYVICDGELAGVFSLSDDCRTGAAEAIRELGSLGIKSVMLTGDSSAAATHAQGQLGGVMEELHSELLPEDKVRLVSGLKARFGPTMMVGDGMNDAAALAAADVGVSMGISGSAAAMETSHATLMSSDVLRVPEAVRLGRCARRTIAVNVAGSVAVKAAVLALAAAWRPVLWAAVLADVGTCLLVVLNSMTLLREEWKGGAKEDGACRATARSLVMRSQLAADSQAPNAADAGAAGREQTNGCRCCPKPGMSPEHSVVIDIRADGERQEERPAEAAVVAKCCGGGGGEGIRCGASKKPTATVVVAKCCGGGGGGEGTRCGASKNPATAAVVAKCCSGGGGEGIGCGASKKPTATAVVAKCCGGGGEGTRCAASKKPATAAVVAKCCGGDGGEGTGCGASKRSPPAEGSCSGGEGGTNGVGRCCTSVKRPTCCDMGAAEVSDSSPETAKDCRNGRCCAKTMNSGEVKG

(19) United States
(12) Patent Application Publication
(10) Pub. No.: US 2025/0388917 A1
(43) Pub. Date: Dec. 25, 2025

(54) GENERATION OF LOW-ARSENIC AND LOW-CADMIUM RICE

(71) Applicant: Temasek Life Sciences Laboratory Limited, Singapore (SG)

(72) Inventors: Yuejing GUI, Singapore (SG); Zhongchao YIN, Singapore (SG)

(73) Assignee: Temasek Life Sciences Laboratory Limited, Singapore (SG)

(21) Appl. No.: 19/140,325

(22) PCT Filed: Dec. 20, 2023
(86) PCT No.: PCT/SG2023/050847

§ 371 Date: Jun. 17, 2025

(30) Foreign Application Priority Data
Dec. 20, 2022 (SG) 10202260511R

(51) Int. Cl. C12N 15/82; C12N 9/00; C12N 9/10

(52) U.S. Cl. CPC C12N 15/8242; C12N 9/00; C12N 9/104; C12Y 203/02015

(57) ABSTRACT

The present invention relates to a genetically modified rice plant or plant cell, comprising a heterologous heavy metal ATPase gene operably linked to an OsActin1 promoter: a heterologous ATP-binding cassette (ABC) transporter gene operably linked to an OsActin1 promoter, and a heterologous phytochelatin synthase gene operably linked to an OsActin1 promoter, wherein the OsActin1 promoter has low activity in a seed endosperm of the modified rice plant compared to its activity in other vegetative tissues of the modified rice plant; wherein a rice grain of said genetically modified rice plant has reduced arsenic (As) and cadmium (Cd) compared to a control rice plant that has not undergone said genetic modification. The present invention also relates to a method of creating such a genetically modified rice plant or plant cell, and a kit for doing so.

### FIELD OF THE INVENTION

The present invention relates to the production of transgenic rice plants genetically modified to contain reduced arsenic and cadmium levels in rice grains compared to the level in control, non-transgenic, rice grains, methods for making such plants, and a bacterium for transforming rice plants into such plants. More particularly, the genetically modified plant of the invention overexpress a heterologous P_{1B}-type heavy metal ATPase gene operably linked to an OsActin1 promoter; a heterologous ATP-binding cassette (ABC) transporter gene operably linked to an OsActin1 promoter, and a heterologous phytochelatin synthase gene operably linked to an OsActin1 promoter, wherein the OsActin1 promoter has low activity in a seed endosperm of the modified rice plant compared to its activity in other vegetative tissues of the modified rice plant.

### BACKGROUND OF THE INVENTION

Arsenic (As) is a highly toxic metalloid that is classified a non-threshold class-1 carcinogen, causing acute and chronic adverse health effects on humans (Hughes, 2002). Cadmium (Cd) is a toxic heavy metal that is preferentially accumulated in livers and kidneys of humans resulting in tubular renal dysfunction, osteoporosis, cardiovascular disease and cancer (Fowler, 2009). Human beings are exposed to As and Cd mainly through water and food that are contaminated with the two toxic elements. Rice (Oryza sativa), feeding more than half the world's population, is a major dietary source of Cd and As. Compared with other cereal crops, rice is likely more efficient at taking up As and Cd from soil, which is attributed to the efficient uptake and transportation systems for Cd and As and the higher bio-accessibility of arsenite [As(III)] as the dominant form in flooded paddy fields (Ma et al., 2008; Sui et al., 2018; Xu et al., 2008). In Bangladesh and India, the As-contaminated groundwater for irrigation of crops results in rice being a major exposure source of As, contributing to around 50% of the total As intake (Panda et al., 2010). In Japan and China, rice was the most influential source of dietary Cd intake among general population (Song et al., 2017; Tsukahara et al., 2003). Thus, the development and production of low As and low Cd rice is always desired for the health of the general population.

Upregulation of the genes involved in As or Cd sequestration is an important and effective approach to generate low As or low Cd rice grains. OsHMA3 is a tonoplast transporter belonging to the P1B-type Heavy Metal ATPase family (Miyadate et al., 2011; Ueno et al., 2010). OsHMA3 is involved in transport of Cd from cytosol into vacuoles in root cells for sequestration, thereby restricting Cd transport from roots to shoots, mitigating Cd toxicity to the aerial parts of rice plants and reducing Cd accumulation in rice grain (Miyadate et al., 2011; Ueno et al., 2010). OsHMA2 is another P_{1B}-Type Heavy Metal ATPase that is localized on plasma membrane as an influx transporter mainly expressed in vascular bundles in roots and nodes (Takahashi et al., 2012; Yamaji et al., 2013). OsHMA2 is involved in transport of zinc (Zn) and Cd through the phloem to the developing tissues (Takahashi et al., 2012; Yamaji et al., 2013). Overexpression of OsHMA3 under the control of the maize ubiquitin gene promoter or the rice OsHMA2 promoter selectively increased Cd sequestration into the root vacuoles and reduced Cd translocation to the rice shoot and grains (Shao et al., 2018; Ueno et al., 2010). In rice, OsABCC1 is a tonoplast transporter that plays an important role in As detoxification. The OsABCC1 knockout mutant shows hypersensitivity to As treatment and has an increased As concentration in rice grain (Hayashi et al., 2017; Song et al., 2014). In cytosol, phytochelatins (PCs) chelate As(III) to form PC—As(III) complexes, which are then transported into vacuoles for sequestration via OsABCC1 (Hayashi et al., 2017; Song et al., 2014). PCs are noncoded, heavy metal (loid) binding peptides with the general structure of (y-Glu-Cys) n (2-11)-Gly (Grill et al., 1985). PCs are synthesized from glutathione (GSH) by phytochelatin synthases (PCSs) (Ha et al., 1999). Two PCSs, OsPCS1 and OsPCS2, have been identified in rice (Das et al., 2017; Hayashi et al., 2017; Li et al., 2007). Overexpression of OsPCS1 was found to enhance PC-dependent As sequestration and significantly reduce the As accumulation in rice grain (Hayashi et al., 2017). In a more sophisticated study, lower grain As rice was achieved by the co-expression of two different vascular As sequestration genes, ScYCF1 (Saccharomyces cerevisiae yeast cadmium factor) and OsABCC1, under the control of the RCc3 (rice root-specific cDNA clone 3) promoter, along with c-ECS (a bacterial c-glutamylcysteine synthetase gene) driven by the maize ubiquitin gene promoter (Deng et al., 2018). However, in another report, heterologous expression of the wheat phytochelatin synthase gene (TaPCS1) in rice resulted in enhanced Cd sensitivity (Wang et al., 2012). Thus, it is necessary to tackle the contradiction for utilizing PCSs to control accumulation of As and Cd in rice grain. More importantly, it was noted that, so far, no study has been reported to focus on reducing As and Cd in rice grain simultaneously by genetic engineering approaches.

The rice actin 1 gene (OsActin1) promoter was found to be highly active in both vegetative and reproductive tissues (Park et al., 2010). However, it was noted that, in developing seeds, the activity of the OsActin1 promoter was mainly detected in aleurones and the embryos but not in the starchy endosperm (Park et al., 2010), which might have potential of driving gene overexpression mainly in vegetative tissues. In this invention, we reported the generation and characterization of transgenic rice lines overexpressing or co-overexpressing OsPCS1, OsABCC1 and OsHMA3 genes under the control of the OsActin1 promoter. Our objective was to generate low-As and low-Cd rice grains by enhancing As and Cd sequestration in vacuoles of cells in vegetative tissues and organs without any pleiotropic phenotypes on transgenic plants or yield penalty.

### SUMMARY OF THE INVENTION

In this disclosure, a strategy is provided for regulating As and Cd allocation in rice, consequently, greatly reduce As and Cd accumulation in grain simultaneously, but did not impair agronomic traits. Phytochelatins (PCs) that chelate heavy metal (liod) play an important role in detoxification of Cd and As in plants. Co-overexpression of the vacuolar PCs—As transporter gene OsABCC1 and the PCs synthase gene OsPCS1 showed a synergistic effect on reducing As concentration in rice grain and increasing As tolerance, leading to maximization of As sequestration. Overexpression of OsPCS1 conferred rice plant As tolerance but resulted in Cd hypersensitivity. When exposed to Cd, the Cd concentration in vacuoles isolated from OsPCS1 transgenic plants was much lower than that of T5105 control plants. An assay of the transport activities for Cd and PC₂—Cd in mesophyll protoplasts and vacuoles from T5105 and OsHMA3 transgenic plants suggested that PC₂—Cd blocked the Cd sequestration into vacuoles via OsHMA3. Contemporaneous overexpression of OsHMA3 and OsPCS1 completely rescued growth defects due to Cd hypersensitivity caused by OsPCS1 overexpression, suggesting OsHMA3 competes with PCs for binding to Cd. Finally, triple overexpression of OsABCC1, OsPCS1 and OsHMA3 under the control of OsActin1 promoter in rice plants exhibited a 92% reduction of As concentration and a 98% reduction of Cd concentration in the brown rice compared with T5105 plants, but did not affected plant growth and essential elements homeostasis. This strategy should be applicable to reduce As and Cd dietary intake through rice consumption.

According to a first aspect, the present invention provides a genetically modified rice plant or plant cell, comprising a heterologous P18-type heavy metal ATPase gene operably linked to an OsActin1 promoter; a heterologous ATP-binding cassette (ABC) transporter gene operably linked to an OsActin1 promoter, and a heterologous phytochelatin synthase gene operably linked to an OsActin1 promoter, wherein the OsActin1 promoter has low activity in a seed endosperm of the modified rice plant compared to its activity in other vegetative tissues of the modified rice plant; wherein a rice grain of said genetically modified rice plant has reduced arsenic (As) and cadmium (Cd) compared to a control rice plant that has not undergone said genetic modification.

In some embodiments, the OsActin1 promoter comprises the nucleic acid sequence set forth in SEQ ID NO: 21 or 31 or a functional sequence variant thereof.

In some embodiments, the heterologous P1B-type heavy metal ATPase gene encodes the amino acid sequence set forth in SEQ ID NO: 39; the heterologous ABC transporter gene encodes the amino acid sequence set forth in SEQ ID NO: 37, and the phytochelatin synthase gene encodes the amino acid sequence set forth in SEQ ID NO: 38.

In some embodiments, the heterologous P18-type heavy metal ATPase gene comprises a nucleic acid sequence having at least 80% sequence identity, at least 85% sequence identity, at least 90% sequence identity, at least 95% sequence identity or 100% sequence identity, due to the degeneracy of the genetic code, to the polynucleotide sequence set forth in SEQ ID NO: 22, the heterologous ABC transporter gene comprises a nucleic acid sequence having at least 80% sequence identity, at least 85% sequence identity, at least 90% sequence identity, at least 95% sequence identity or 100% sequence identity, due to the degeneracy of the genetic code, to the polynucleotide sequence set forth in SEQ ID NO: 36, and the heterologous phytochelatin synthase gene comprises a nucleic acid sequence having at least 80% sequence identity, at least 85% sequence identity, at least 90% sequence identity, at least 95% sequence identity or 100% sequence identity, due to the degeneracy of the genetic code, to the polynucleotide sequence set forth in SEQ ID NO: 32.

In some embodiments, the exogenous P_{1B}-typ heavy metal ATPase gene, the exogenous ABC transporter gene and/or the exogenous phytochelatin synthase gene are from a cereal crop.

In some embodiments, the heterologous P_{1B}-typ heavy metal ATPase gene is OsHMA3 and comprises the nucleic acid sequence set forth in SEQ ID NO: 22, the heterologous ABC transporter gene is OsABCC1 and comprises the nucleic acid sequence set forth in SEQ ID NO: 36, and the heterologous phytochelatin synthase gene is OsPCS1 and comprises the nucleic acid sequence set forth in SEQ ID NO: 32.

In some embodiments, the genetically modified rice plant or plant cell comprises a heterologous OsHMA3 gene operably linked to an OsActin1 promoter, a heterologous OsABCC1 gene operably linked to an OsActin1 promoter, and a heterologous OsPCS1 gene operably linked to an OsActin1 promoter.

In some embodiments, the genetically modified rice plant or plant cell is the species Oryza sativa L.

According to a second aspect, the present invention provides a method of creating a genetically modified rice plant, that has reduced arsenic (As) and cadmium (Cd) in its rice grain compared to a rice grain from a control rice plant, the method comprising the steps of:
a) generating a genetically modified rice plant comprising a heterologous P_{1B}-type heavy metal ATPase gene operably linked to an OsActin1 promoter;
b) generating a genetically modified rice plant comprising a heterologous ATP-binding cassette (ABC) transporter gene operably linked to an OsActin1 promoter;
c) generating a genetically modified rice plant comprising a heterologous phytochelatin synthase gene operably linked to an OsActin1 promoter;
d) select genetically modified rice plants which, respectively, overexpress said exogenous P_{1B}-type heavy metal ATPase gene, said exogenous ATP-binding cassette (ABC) transporter gene and said exogenous phytochelatin synthase gene;
e) crossing two of the three genetically modified rice plants to generate double homozygote plants with respect to the exogenous genes; and
f) crossing a double homozygote plant from step (e) with a third genetically modified rice plant to generate triple homozygote plants that overexpress the exogenous genes.

In some embodiments, the OsActin1 promoter is as defined in the first aspect.

In some embodiments of the method according to the second aspect, the heterologous genes are as defined in the first aspect.

According to a second aspect, the present invention provides a kit for creating a genetically modified rice plant, having reduced arsenic (As) and cadmium (Cd) in its rice grain compared to a rice grain from a control rice plant, wherein the kit comprises bacteria containing vectors comprising a heterologous heavy metal ATPase gene operably linked to an OsActin1 promoter and/or bacteria containing vectors comprising a heterologous ATP-binding cassette (ABC) transporter gene operably linked to an OsActin1 promoter and/or bacteria containing vectors comprising a heterologous phytochelatin synthase gene operably linked to an OsActin1 promoter. In preferred embodiments, the bacteria is Agrobacterium tumefaciens.

In some embodiments, the OsActin1 promoter comprises the nucleic acid sequence set forth in SEQ ID NO: 21 or 31 or a functional sequence variant thereof.

In some embodiments, the heterologous P_{1B}-type heavy metal ATPase gene encodes the amino acid sequence set forth in SEQ ID NO: 39; the heterologous ABC transporter gene encodes the amino acid sequence set forth in SEQ ID NO: 37, and the phytochelatin synthase gene encodes the amino acid sequence set forth in SEQ ID NO: 38.

In some embodiments, the heterologous P_{1B}-type heavy metal ATPase gene comprises a nucleic acid sequence having at least 80% sequence identity, at least 85% sequence identity, at least 90% sequence identity, at least 95% sequence identity or 100% sequence identity, due to the degeneracy of the genetic code, to the polynucleotide sequence set forth in SEQ ID NO: 22, the heterologous ABC transporter gene comprises a nucleic acid sequence having at least 80% sequence identity, at least 85% sequence identity, at least 90% sequence identity, at least 95% sequence identity or 100% sequence identity, due to the degeneracy of the genetic code, to the polynucleotide sequence set forth in SEQ ID NO: 36, and the heterologous phytochelatin synthase gene comprises a nucleic acid sequence having at least 80% sequence identity, at least 85% sequence identity, at least 90% sequence identity, at least 95% sequence identity or 100% sequence identity, due to the degeneracy of the genetic code, to the polynucleotide sequence set forth in SEQ ID NO: 32.

In some embodiments, the exogenous P_{1B}-typ heavy metal ATPase gene, the exogenous ABC transporter gene and/or the exogenous phytochelatin synthase gene are from a cereal crop.

In some embodiments, the heterologous P_{1B}-typ heavy metal ATPase gene is OsHMA3 and comprises the nucleic acid sequence set forth in SEQ ID NO: 22, the heterologous ABC transporter gene is OsABCC1 and comprises the nucleic acid sequence set forth in SEQ ID NO: 36, and the heterologous phytochelatin synthase gene is OsPCS1 and comprises the nucleic acid sequence set forth in SEQ ID NO: 32.

### BRIEF DESCRIPTION OF THE FIGURES

FIG. 1 shows a diagram of the binary constructs and genes used in this study. (A) Schematic map of binary constructs. The lower panel of the diagram shows the genes in the T-DNA regions of binary constructs with backbone derived from pCAMBIA1305.1. The upper panel of the diagram shows the genes of interest that were used to replace GUSPlus in pCAMBIA1305.1. The cDNA clones of the OsHMA3 and OsPCS1 genes were used to make constructs pCActin1-cHMA3 and pCActin1-cPCS1, respectively, whereas the genomic clone of the OsABCC1 gene was used to make construct pCActin1-gABCC1. In all constructs, the CaMV35S promoter in pCAMBIA1305.1 was replaced with OsActin1 promoter. The diagram was not drawn to scale. (B) Contig map of pCActin1-gABCC1. The contig map was generated with Sequencher 5.1 (Gene Codes Corporation, MI 48108, USA). It starts from the left border to the right border of T-DNA followed by pC1305.1 backbone region. pCActin1-gABCC1 harbors the hygromycin phosphotransferase gene (P_{35S}:Hpt:T_{35S}) and the genomic clone of the OsABCC1 gene under the control of OsActin1 gene promoter (P_{Actin1}:gABCC1:T_{Nos}) in the T-DNA region as shown in (A). The P_{35S}:Hpt:T_{35S} gene has the transcription orientation towards LB, while the P_{Actin1}:gABCC1:T_{Nos} gene has the transcription orientation towards RB. (C) Contig map of pCActin1-cPCS1. The contig map was generated with Sequencher 5.1 (Gene Codes Corporation, MI 48108, USA). It starts from the left border to the right border of T-DNA followed by pC1305.1 backbone region. pCActin1-cPCS1 harbors the hygromycin phosphotransferase gene (P_{35S}:Hpt:T_{35S}) and the cDNA clone of the OsPCS1 gene under the control of OsActin1 gene promoter (P_{Actin1}:cPCS1:T_{Nos}) in the T-DNA region as shown in (A). The P_{35S}:Hpt:T_{35S} gene has the transcription orientation towards LB, while the P_{Actin1}:cPCS1:T_{Nos} gene has the transcription orientation towards RB. (D) Contig map of pCActin1-cHMA3. The contig map was generated with Sequencher 5.1 (Gene Codes Corporation, MI 48108, USA). It starts from the left border to the right border of T-DNA followed by pC1305.1 backbone region. pCActin1-cHMA3 harbors the hygromycin phosphotransferase gene (P_{35S}:Hpt:T_{35S}) and the cDNA clone of the OsHMA3 gene under the control of OsActin1 gene promoter (P_{Actin1}:cHMA3:T_{Nos}) in the T-DNA region as shown in (A). The P_{35S}:Hpt:T_{35S} gene has the transcription orientation towards LB, while the P_{Actin1}:cHMA3:T_{Nos} gene has the transcription orientation towards RB. Abbreviations in (A) to (D): LB, left border; T_{35S}, CaMV35S terminator; Hpt CDS, the coding region of the hygromycin phosphotransferase gene; P_{35S}, CaMV35S promoter; P_{Actin1}, OsActin1 promoter; GOI, gene-of-interest; T_{Nos}, nopaline synthase gene terminator; RB, Right border; ORF, open reading frame

FIG. 2 shows results on generation of OsHMA3-overexpressed lines. (A) Detection of the copy number of T-DNA in transgenic P_{Actin1}:cHAM3:T_{Nos} plants (TO) by Southern blot analysis. M, molecular marker. (B) Expression level of the OsHMA3 gene in T5105 and transgenic P_{Actin1}:cHAM3:T_{Nos} plants (T0) detected by qRT-PCR. Only the T0 plants that harboured a single copy of the P_{Actin1}:CHAM3:T_{Nos} gene were selected and examined. (C) Morphological phenotype of T5105 and OsHMA3 over-expressing line HMA3-L3 (T2). Plants were imaged at 120 d after sowing. (D) and (E) Plant height (D) and seed setting rates (E) of T5105 and OsHMA3 over-expressing lines (T2). Values are means ±SD with three biological replicates. No significant difference was observed between T5105 and OsHMA3 over-expressing lines (P>0.05 by Student's t test). L1, HMA3-L1; L3, HMA3-L3 and L12, HMA3-L12.

FIG. 3 shows results on generation of OsABCC1-overexpressed lines. (A) Detection of the copy number of T-DNA in transgenic P_{Actin1}:gABCC1:T_{Nos} plants (TO) by Southern blot analysis. M, molecular marker. (B) Expression level of the OsABCC1 gene in T5105 (NT) and transgenic P_{Actin1}:gABCC1:T_{Nos} plants (TO) detected by qRT-PCR. Only the TO plants that harboured a single copy of the P_{Actin1}:gABCC1:T_{Nos} gene were selected and examined. (C) Morphological phenotype of T5105 and OsABCC1 over-expressing line ABCC1-L27 (T2). Plants were imaged at 120 d after sowing. (D) and (E) Plant height (D) and seed setting rates (E) of T5105 and OsABCC1 over-expressing lines (T2). Values are means ±SD with three biological replicates. No significant difference was observed between T5105 and OsABCC1 over-expressing lines (P>0.05 by Student's t test). L3, ABCC1-L3; L27, ABCC1-L27; L31, ABCC1-L31.

FIG. 4 shows results on generation of OsPCS1-overexpressed lines. (A) Detection of the copy number of T-DNA in transgenic P_{Actin1}:cPCS1:T_{Nos} plants (TO) by Southern blot analysis. M, molecular marker. (B) Expression level of the OsPCS1 gene in T5105 (NT) and transgenic P_{Actin1}:cPCS1:T_{Nos} plants (TO) detected by qRT-PCR. Only the TO plants that harboured a single copy of the P_{Actin1}:cPCS1:T_{Nos} gene were selected and examined. (C) Morphological phenotype of T5105 and OsPCS1 over-expressing line PCS1-L1 (T2). Plants were photographed at 120 d after sowing. (D) and (E) Plant height (D) and seed setting rates (E) of T5105 and OsPCS1 over-expressing lines (T2). Values are means±SD with three biological replicates. No significant difference was observed between T5105 and OsPCS1 over-expressing lines (P>0.05 by Student's t test). L1, PCS1-L1; L3, PCS1-L3; L4, PCS1-L4.

FIG. 5 shows the results on characterization of OsHMA3-overexpressed lines. (A) Morphology of the panicles of T5105 and three independent OsHMA3-overexpressed lines. (B) Expression level of OsHMA3 in T5105 and the OsHMA3-overexpressed lines. (C) The Cd concentration in grains of T5105 and the OsHMA3-overexpressed lines grown in soils with or without Cd treatment. (D) The Cd concentration in different straw tissues of T5105 and the OsHMA3-overexpressed line HMA3-L3 grown in soils with Cd treatment. (E) Seedlings of T5105 (NT) and the OsHMA3-overexpressed lines at 14 d after Cd treatment. (F) and (G) Shoot length (F) and dry weight (DW) (G) of T5105 and the OsHMA3-overexpressed lines at 14 d after Cd treatment. Data are means±SD with three biological replicates. The significant differences between T5105 and the OsHMA3-overexpressed lines were calculated using a Student's t test (*P<0.05; ** P<0.01). Control, soil without Cd treatment; Control+Cd, soil containing 3 mg/kg Cd in the form of CdSO₄.

FIG. 6 shows the results on characterization of OsABCC1-overexpressed lines. (A) Morphology of the panicles of T5105 and the OsABCC1-overexpressed lines. (B) Expression levels of OsABCC1 in T5105 and the OsABCC1-overexpressed lines. (C) The As concentration in grains of T5105 and the OsABCC1-overexpressed lines grown in soils with or without As treatment. (D) The As concentration in different straw tissues of T5105 and the OsABCC1-overexpressed line ABCC1-L27 grown in soils with As treatment. (E) Seedlings of T5105 (NT) and the OsABCC1-overexpressed lines at 14 d after As treatment. (F) and (G) Shoot length (F) and dry weight (DW) (G) of T5105 and the OsABCC1-overexpressed lines at 14 d after As treatment. All data are means±SD of at least three biological replicates. The significant differences between T5105 and the OsABCC1-overexpressed lines were calculated using a Student's t test (*P<0.05; ** P<0.01). Control, soil without As treatment; Control+As, soil containing 10 mg/kg As in the form of NaAsO₂.

FIG. 7 shows test results of T5105 and the OsABCC1-overexpressed lines for tolerance to Cd treatment. (A) Seedlings of T5105 (NT) and the OsABCC1-overexpressed lines at 14 d after treatment on Cd-containing medium. NT, T5105; L3, ABCC1-L3; L27, ABCC1-L27; L31, ABCC11-L31. (B) and (C) Shoot length (B) and dry weight (DW) (C) of T5105 and the OsABCC1-overexpressed lines at 14 d after treatment on Cd-containing medium. (D) Cd concentration in the grains of T5105 and ABCC1-L27 grown in soils. Control, soil without Cd treatment; Control+Cd, soil containing 3 mg/kg Cd in the form of CdSO4. Values are means ±SD with three biological replicates. No significant difference was observed between T5105 and the OsABCC1-overexpressed lines (P>0.05 by Student's t test).

FIG. 8 shows the results on characterization of OsPCS1-overexpressed lines. (A) Morphology of the panicles of T5105 and the OsPCS1-overexpressed lines. (B) Expression levels of OsPCS1 in T5105 and the OsAPCS1-overexpressed lines. (C) The As concentration in grains of T5105 and the OsPCS1-overexpressed lines grown in soils with or without As treatment. (D) The Cd concentration in grains of T5105 and the OsPCS1-overexpressed lines grown in soils with or without Cd treatment. (E) and (F) The As (E) or Cd (F) concentration in different straw tissues of T5105 and the OsPCS1-overexpressed line PCS1-L1 grown in soils with As or Cd treatment. All data are means±SD of at least three biological replicates. The significant differences between T5105 and the OsPCS1-overexpressed lines were calculated using a Student's t test (*P<0.05; ** P<0.01). Control, soil without As or Cd treatment; Control+As, soil containing 10 mg/kg As in the form of NaAsO2. Control+Cd, soil containing 3 mg/kg Cd in the form of CdSO₄.

FIG. 9 shows test results of OsPCS1-overexpressed lines for As and Cd tolerance. (A) Seedlings of T5105 (NT) and the OsPCS1-overexpressed lines at 14 d after As treatment. (B) and (C) Shoot length (B) and dry weight (DW) (C) of T5105 and the OsPCS1-overexpressed lines at 14 d after As treatment. (D) Seedlings of T5105 (NT) and the OsPCS1-overexpressed lines at 14 d after Cd treatment. (E) and (F) Shoot length (E) and dry weight (DW) (F) of T5105 and the OsPCS1-overexpressed lines at 14 d after Cd treatment. All data are means±SD with three biological replicates. The significant differences between T5105 and the OsPCS1-overexpressed lines were calculated using a Student's t test (*P<0.05; ** P<0.01). NT, T5105; L1, PCS1-L1; L3, PCS1-L3; L4, PCS1-L4.

FIG. 10 shows test results of OsABCC1 and OsPCS1 co-overexpressed line for As tolerance. (A) Seedlings of T5105 (NT), ABCC1-L27 (A27), PCS1-L1 (P1) and OsABCC1 and OsPCS1 co-overexpressed line (AP) at 14 d after As treatment. (B) and (C) Shoot length (B) and dry weight (DW) (C) of rice plants as shown in (A). (D) and (E) As concentration in roots (D) and shoots (E) of rice plants as shown in (A). (F) The As concentration in grains of T5105, ABCC1-L27, PCS1-L1 and AP plants grown in soils with or without As treatment. Data are means±SD with three biological replicates. Different letters indicate significant difference calculated by one-way ANOVA followed by LSD's test at P<0.05. Control, soil without As treatment; Control+As, soil containing 10 mg/kg As in the form of NaAsO₂.

FIG. 11 shows test results of OsPCS1 and OsHMA3 co-overexpressed line for Cd tolerance. (A) Seedlings of T5105 (NT), PCS1-L1 (P1), HMA3-L3 (H3) and OsHMA3 and OsPCS1 co-overexpressed line (HP) at 14 d after Cd treatment. (B) and (C) Shoot length (B) and dry weight (DW) (C) of rice plants as shown in (A). (D) and (E) As concentration in roots (D) and shoots (E) of rice plants as shown in (A). (F) The Cd concentration in grains of T5105, HMA3-L3, PCS1-L1 and HP plants grown in soils with or without Cd treatment. Data are means±SD with three biological replicates. Different letters indicate significant difference calculated by one-way ANOVA followed by LSD's test at P<0.05. Control, soil without Cd treatment; Control+Cd, soil containing 3 mg/kg Cd in the form of CdSO₄.

FIG. 12 shows test results of the effect of OsPCS1 overexpression and phytochelatin 2-Cd complex (PC2-Cd) on OsHMA3-mediated Cd sequestration in vacuoles. (A) Cd concentration in vacuoles and protoplasts isolated from T5105 and PCS1-L1 seedlings grown on Cd-containing medium. The significant differences between T5105 and the OsPCS1-L1 were calculated using a Student's t test (** P<0.01). Data are means±SD with three biological replicates. (B) Cd concentration in vacuoles and protoplasts of T5105 and HMA3-L3 after incubation of protoplasts with Cd or PC2-Cd complex. The significant differences between Cd treatment and PC2-Cd treatment were calculated using a Student's t test (** P<0.01). Data are means±SD with three biological replicates.

FIG. 13 shows As and Cd concentration in the grains of the OsABCC1, OsPCS1 and OsHMA3 co-overexpressed line. (A) Morphological phenotype of T5105 and the OsABCC1, OsPCS1 and OsHMA3 co-expressing line (PAH). Plants were photographed at 120 d after sowing. (B) Plant height of T5105 and PAH plants. (C) Panicles and unpolished rice grains of T5105 and PAH plants. (D) Seed setting rate of T5105 and PAH plants. (E) Weight of 100 grains of T5105 and PAH plants. Data are means±SD with at least three biological replicates in (B), (D) and (E). No significant difference was detected between T5105 and PAH plants using a Student's t test (P>0.05) (F) and (G) As (F) and Cd (G) concentration in the rice grain of T5105, PCS1-L1, ABCC1-L27, HMA3-L3 and PAH plants grown in soils with or without double treatment with As and Cd. Data are means±SD with three biological replicates. Different letters in (F) and (G) indicate significant difference calculated by one-way ANOVA followed by LSD's test at P<0.05. Control, soil without As or Cd treatment; Control+As+Cd, soil containing 10 mg/kg As in the form of NaAsO2 and 3 mg/kg Cd in the form of CdSO₄.

FIG. 14 shows concentrations of other elements in the grains of T5105 and transgenic plants. T5105, PCS1-L1, ABCC1-L27, HMA3-L3 and PAH plants were grown in soils with or without double treatment with As and Cd. The concentration of Co, Cu, Fe, Mn, Se and Zn in the grain of were determined by ICP-MS. Data are means±SD with three biological replicates. No significant difference was detected between T5105 and transgenic plants by LSD's test at P>0.05. Control, soil without As or Cd treatment; Control+As+Cd, soil containing 10 mg/kg As in the form of NaAsO₂ and 3 mg/kg Cd in the form of CdSO₄.

FIG. 15 shows test results of T5105, PCS1-L1, ABCC1-L27, HMA3-L3 and PAH plants for tolerance to As and Cd. (A) Seedlings of T5105, PCS1-L1 (P1), ABCC1-L27 (A27), HMA3-L3 (H3) and PAH plants at 14 d after treatment with As and Cd. (B) and (C) Shoot length (B) and dry weight (DW) (C) of rice plants as shown in (A). (D) and (E) As concentration in roots (D) and shoots (E) of rice plants as shown in (A). (F) and (G) Cd concentration in roots (F) and shoots (G) of rice plants as shown in (A). Data are means±SD with three biological replicates. Different letters indicate significant difference calculated by one-way ANOVA followed by LSD's test at P<0.05. As+Cd Treatment 1 (As+Cd T1), half-strength MS medium containing 50 μM NaAsO₂+10 μM CdSO₄; As+Cd Treatment 2 (As+Cd T2), half-strength MS medium containing 75 μM NaAsO₂ and 20 μM CdSO₄.

### DETAILED DESCRIPTION OF THE INVENTION

Bibliographic references mentioned in the present specification are for convenience listed in the form of a list of references and added at the end of the examples. The whole content of such bibliographic references is herein incorporated by reference.

### Definitions

For convenience, certain terms employed in the specification, examples and appended claims are collected here.

The term “comprising” is herein defined to be that where the various components, ingredients, or steps, can be conjointly employed in practising the present invention. Accordingly, the term “comprising” encompasses the more restrictive terms “consisting essentially of” and “consisting of.”

The term “Agrobacterium” refers to a soil-borne, Gram-negative, rod-shaped phytopathogenic bacterium which causes crown gall. The term “Agrobacterium” includes, but is not limited to, the strains Agrobacterium tumefaciens (which typically causes crown gall in infected plants), and Agrobacterium rhizogens (which causes hairy root disease in infected host plants). Infection of a plant cell with Agrobacterium generally results in the production of opines (e.g., nopaline, agropine, octopine etc.) by the infected cell.

The term “expression” when used in reference to a nucleic acid sequence, such as a gene, refers to the process of converting genetic information encoded in a gene into RNA (e.g., mRNA, rRNA, tRNA, or snRNA) through “transcription” of the gene (i.e., via the enzymatic action of an RNA polymerase), and into protein where applicable (as when a gene encodes a protein), through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively. “Overexpression” refers to an expression level higher than what would normally be observed for a particular gene.

The terms “nucleic acid sequence,” “nucleotide sequence of interest” or “nucleic acid sequence of interest” refer to any nucleotide sequence (e.g., RNA or DNA), the manipulation of which may be deemed desirable for any reason (e.g., treat disease, confer improved qualities, etc.), by one of ordinary skill in the art. Such nucleotide sequences include, but are not limited to, coding sequences of structural genes (e.g., reporter genes, selection marker genes, oncogenes, drug resistance genes, growth factors, etc.), and non-coding regulatory sequences which do not encode an mRNA or protein product (e.g., promoter sequence, polyadenylation sequence, termination sequence, enhancer sequence, etc.).

The terms “amino acid” or “amino acid sequence,” as used herein, refer to an oligopeptide, peptide, polypeptide, or protein sequence, or a fragment of any of these, and to naturally occurring or synthetic molecules. Where “amino acid sequence” is recited herein to 15 refer to an amino acid sequence of a naturally occurring protein molecule, “amino acid sequence” and like terms are not meant to limit the amino acid sequence to the complete native amino acid sequence associated with the recited protein molecule.

As used herein, the term “functional sequence variant” refers to a polynucleotide sequence (reference or wild-type sequence) that is altered by one or more nucleic acids without abolishing or substantially altering the polynucleotide activity of the non-variant reference. For example, the OsActin1 promoter defined by SEQ ID NO: 21 or SEQ ID NO: 31 may be truncated or have one or more nucleic acids removed internally and retain activity.

The term “gene” encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region termed “exon” or “expressed regions” or “expressed sequences” interrupted with non-coding sequences termed “introns” or “intervening regions” or “intervening sequences.”

The term “seed” as used herein includes all tissues that result from the development of a fertilized plant egg; thus, it includes a matured ovule containing an embryo and stored nutrients, as well as an integument or integuments that differentiated into a protective seed coat or testa. The nutrients in seed tissues may be stored in the endosperm or in the body of the embryo, notably in the cotyledons, or both.

As used herein, the term “seed” may also refer to a mature and fertilized, i.e. ripened, ovule of a seed plant comprising a plant embryo (i.e. miniature plant) and further comprising an endosperm (i.e. supply of food for the plant embryo) and may be enclosed by a seed coat.

As used herein, the term “rice” in reference to a “rice plant” is an Oryza spp., i.e. cultivated varieties, noncultivated rice plants and ancestral rice plants. Preferably the rice plant of the invention is an Oryza sativa variety.

The term “heterologous” when used in reference to a gene or nucleic acid refers to a gene that has been manipulated in some way. For example, a heterologous gene includes a gene from one species introduced into another species. A heterologous gene also includes a gene native to an organism that has been altered in some way (e.g., mutated, added in multiple copies, linked to a non-native promoter or enhancer sequence, etc.). Heterologous genes may comprise plant gene sequences that comprise cDNA forms of a plant gene; the cDNA sequences may be expressed in either a sense (to produce mRNA) or anti-sense orientation (to produce an anti-sense RNA transcript that is complementary to the mRNA transcript). Heterologous genes are distinguished from endogenous plant genes in that the heterologous gene sequences are typically joined to nucleotide sequences comprising regulatory elements such as promoters that are not found naturally associated with the gene for the protein encoded by the heterologous gene or with plant gene sequences in the chromosome, or are associated with portions of the chromosome not found in nature (e.g., genes expressed in loci where the gene is not normally expressed).

The term “transgene” refers to a foreign gene that is placed into an organism by the process of transfection. The term “foreign gene” refers to any nucleic acid (e.g., gene sequence) that is introduced into the genome of an organism by experimental manipulations and may include gene sequences found in that organism so long as the introduced gene does not reside in the same location, as does the naturally occurring gene. A transgene may also refer to an “exogenous gene” such that exogenous genes include but are not limited to reporter genes, marker genes, selection genes, and functional genes. The term “endogenous gene” refers to a gene naturally encoded and expressed.

The terms “transformants” and “transformed cells” include the primary transformed cell and cultures derived from that cell without regard to the number of transfers. Resulting progeny may not be precisely identical in DNA content, due to deliberate or inadvertent mutations. Mutant progeny that has the same functionality as screened for in the originally transformed cell are included in the definition of transformants.

The terms “in operable combination”, “in operable order” and “operably linked” refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The terms “overexpressed”, “overexpression” and “overexpressing” and grammatical equivalents, are specifically used in reference to levels of mRNA to indicate a level of expression approximately 3-fold higher than that typically observed in a given tissue in a control or non-transgenic animal. Levels of mRNA are measured using any of a number of techniques known to those skilled in the art including, but not limited to qRT-PCR.

The terms “promoter element,” “promoter,” or “promoter sequence” refer to a DNA sequence that is located at the 5′ end (i.e. precedes) of the coding region of a DNA polymer. The location of most promoters known in nature precedes the transcribed region. The promoter functions as a switch, activating the expression of a gene. If the gene is activated, it is said to be transcribed, or participating in transcription. Transcription involves the synthesis of mRNA from the gene. The promoter, therefore, serves as a transcriptional regulatory element and also provides a site for initiation of transcription of the gene into mRNA.

The term “vector” refers to nucleic acid molecules that transfer DNA segment(s). Transfer can be into a cell, cell to cell, etc. The term “vehicle” is sometimes used interchangeably with “vector.”

The following examples are provided in order to demonstrate and further illustrate certain preferred embodiments and aspects of the present invention and are not to be construed as limiting the scope thereof.

### Example 1

### Materials and Methods

### Plant Materials and Growth Conditions

The rice cultivar used in this study was T5105, an improved aromatic rice in the genetic background of Thai fragrance KTML 105 (Luo and Yin, 2013). Treated or untreated T5105 and transgenic plants were grown in pot soils in a greenhouse at 24-33° C. with a photoperiod of 12 h daylight and 12 h darkness and relative humidity at 80-85%.

### Genes, Constructs and Rice Transformation

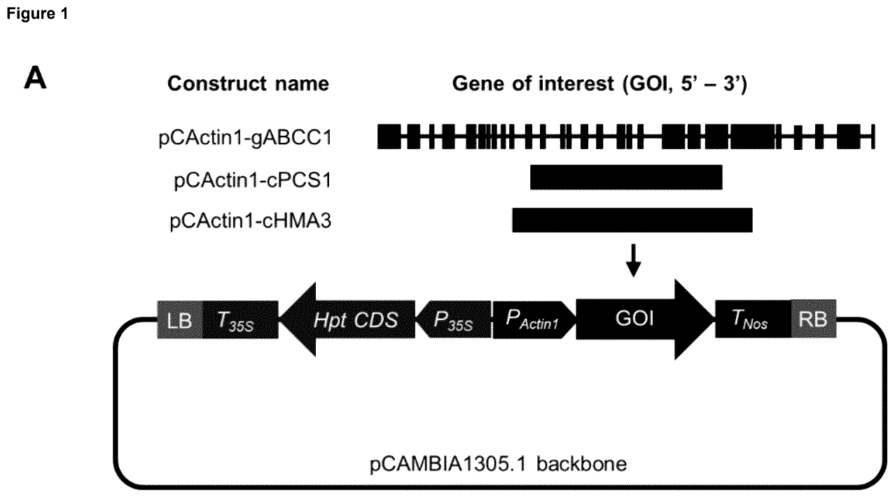

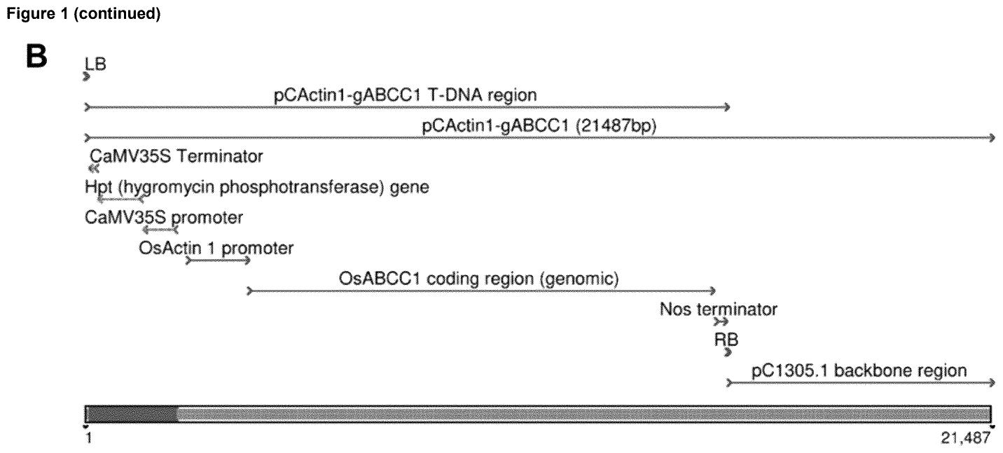

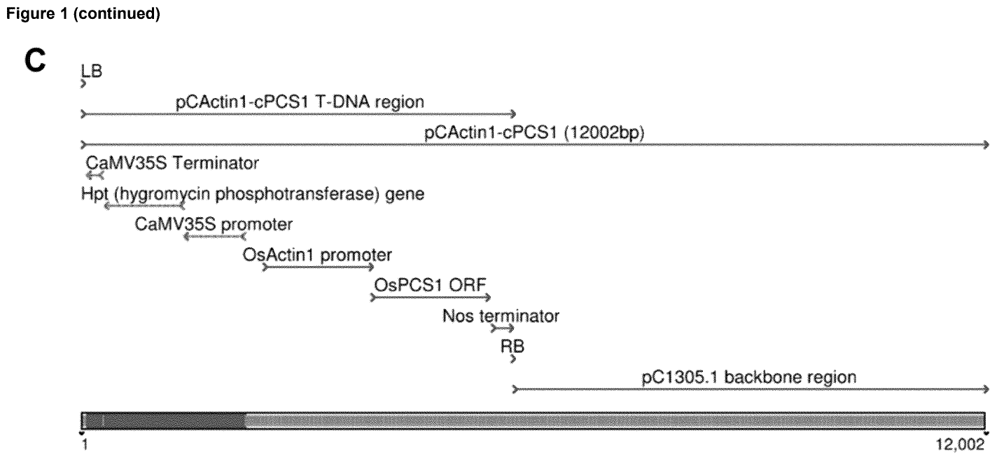

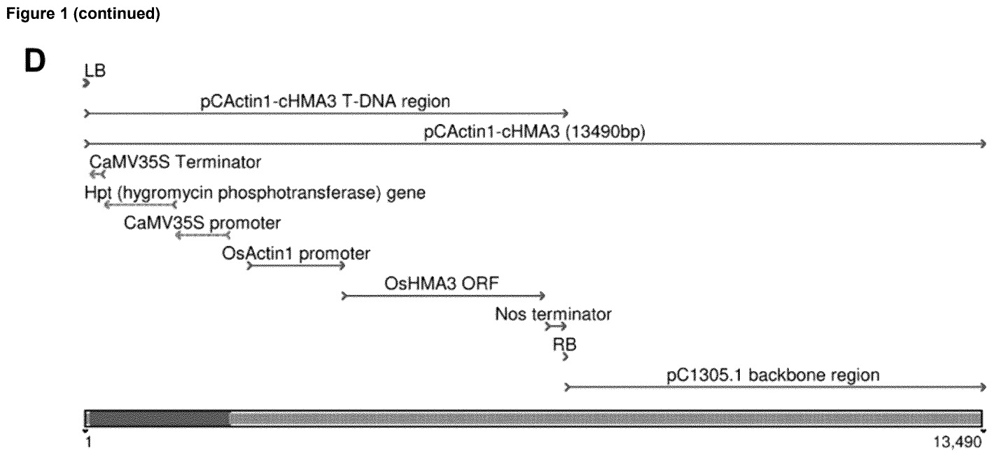

Constructs for gene over-expression in rice was made based on binary vector pCAMBIA1305.1 (Accession no. AF304545) as shown schematically in FIG. 1. Briefly, the CaMV35S promoter at the upstream of the GUSPlus™ gene in the T-DNA region of pCAMBIA1305.1 was replaced by a 1,414-bp promoter (SEQ ID NO: 31 for pCActin1-cPCS1 and pCActin1-gABCC1; SEQ ID NO: 21 for pCActin1-cHMA3) derived from the rice OsActin1 gene (Os03g0718100) (Reece et al., 1990). The cDNA sequence of OsHMA3 (Os07g0232900) (SEQ ID NO: 22) and OsPCS1 (Os05g0415200) (SEQ ID NO: 32) were synthesized by GenScript (www.genscript.com.cn). The full-length genomic DNA fragment (from start codon to stop codon) of OsABCC1 (Os04g0620000) (SEQ ID NO: 35) was amplified by PCR from T5105. The coding region of the GUSPlus™ gene was substituted by the coding regions derived from the cDNA clones of OsHMA3 (Os07g0232900) (SEQ ID NO: 22), OsPCS1 (Os05g0415200) (SEQ ID NO: 32) or the genomic clone of OsABCC1 (Os04g0620000) (SEQ ID NO: 35) to yield binary constructs pCActin1-cHMA3, PCActin1-cPCS1 and pCActin1-gABCC1 that harbour promoter fusion genes P_{Actin1}:cHMA3:T_{Nos}, P_{Actin1}:cPCS1:T_{Nos} and P_{Actin1}:gABCC1:T_{Nos}, respectively; as well as a NOS terminator (SEQ ID NO: 23), LB (SEQ ID NO: 27) and RB (SEQ ID NO: 28). All constructs were introduced into the Agrobacterium tumefaciens strains AGL1 and used for rice transformation. The Agrobacterium-mediated transformation of T5105 was performed according to the procedures as described previously with slight modification (Hiei et al., 1994). The 1 mg/L KT and 0.2 mg/L NAA in the rice regeneration medium N6S3-CH were replaced with 1 mg/L BA and 1 mg/L NAA. The gene constructs are shown in FIG. 1A to 1D and SEQ ID NOs: 19-20, 29-30, 33-34.

### Southern Blotting Analysis

Genome DNA from transgenic rice was extracted using E.Z.N.A.® HP plant DNA mini kit (Omega BIO-TEK). About 2 μg DNA was digested with restriction enzymes Hind II and BamH I (NEB). DNA fragments were separated on a 0.8% (w/v) agarose gel by gel electrophoresis. The fragments then were blotted from the agarose gel onto a Hybond-N⁺ membrane (GE Healthcare). Digoxigenin labelled specific nucleic acid probes for hpt gene were amplified by PCR using DIG DNA labelling Mix (Roche) and primer pairs listed in Table 1. Southern blot hybridization and detection of the DIG-labelled probes were performed according to manufacturer's instruction using DIG-High Prime DNA Labeling and Detection Starter Kit II (Roche). ChemiDoc™ Touch imaging system (Bio-Rad) was used to detect the chemiluminescent signal.

TABLE 1
Oligo primers used in this study

| | | | SEQ |
|---|---|---|---|
| Name of primer | DNA sequences (5′ to 3′) | Purpose | ID NO: |
| ABCC1-F | TTGTAGGTAGAAGCCATGGGTTTTGA | Making | 1 |
| | TCCACTG | construct | |
| ABCC1-R | CACCTTACTTTGCCGTCACAAGCTTC | Making | 2 |
| | TACATTTG GTCCCAGTC | construct | |
| ABCC1-qPCR-F | AACAGTGGCTTATGTTCCTCAAG | qRT-qPCR | 3 |
| ABCC1-qPCR-R | AACTCCTCTTTCTCCAATCTCTG | qRT-qPCR | 4 |
| PCS1-qPCR-F | AGCCCAAGTAAAGAGGCTAAC | qRT-qPCR | 5 |
| PCS1-qPCR-R | TACAACAGGGCTGCTTAGAAC | qRT-qPCR | 6 |
| HMA3-qPCR-F | CAGAACAGCAGGTCGAAGAC | qRT-qPCR | 7 |
| HMA3-qPCR-R | CCATTGCTCAAGGCCATCT | qRT-qPCR | 8 |
| EF-qPCR-F | GCACGCTCTTCTTGCTTTC | qRT-PCR | 9 |
| EF-qPCR-R | AGGGAATCTTGTCAGGGTTG | qRT-PCR | 10 |
| HPT-F | AGCCTGAACTCACCGCGACGT | DNA probe | 11 |
| HPT-R | TACTTCTACACAGCCATCGGTCCA | DNA probe | 12 |
| LB-intact-F | CATTGCGGACGTTTTTAATGTAC | Characterizing | 13 |
| | | transgene in | |
| | | transgenic rice | |
| LB-intact-R | TCTCGATGAGCTGATGCTTTGG | Characterizing | 14 |
| | | transgene in | |
| | | transgenic rice | |
| RB-intact-ABCC1-F | GGAAGCACAACACCGAAATTG | Characterizing | 15 |
| | | transgene in | |
| | | transgenic rice | |
| RB-intact-PCS1-F | CACCGATATCCTCGACTGAAAC | Characterizing | 16 |
| | | transgene in | |
| | | transgenic rice | |
| RB-intact-HM3-F | ATGGTGTTGGTCGTTGCT | Characterizing | 17 |
| | | transgene in | |
| | | transgenic rice | |
| RB-intact-R | CCCGATCTAGTAACATAGATG | Characterizing | 18 |
| | | transgene in | |
| | | transgenic rice | |

### Gene Expression Analyses

Total RNA was extracted using a Favorprep™ plant total RNA purification mini kit (FAVORGEN) followed by DNA digestion using DNase I (Roche). The first-strand cDNAs were 5 synthesised from 1 μg of total RNA using cDNA synthesis kit (Bio-Rad). Quantitative real-time PCR (qRT-PCR) was performed on CFX96™ real-time system (Bio-Rad) using SYBR® FAST qPCR Master Mix (KAPA Biosystems). The expression level of the rice elongation factor (EF) gene OsEF-1α (Os03g0178000) gene was used as the internal control. The primers for qRT-PCR of different genes are listed in Table 1.

### Test of Rice Seedlings for Tolerance to as and Cd

Rice seeds were surface-sterilized and germinated on half-strength MS medium in Phytatray™ II vessels (Sigma-Aldrich) at 25° C. in a tissue culture room with a photoperiod of 16 h light and 8 h darkness. Two-week-old rice seedlings were transferred to half-strength MS medium containing different concentrations of NaAsO₂ (0-100 UM) and/or CdSO₄ (0-40 μM) and cultured for another 14 d. The roots of treated seedlings were washed for three times with 5 mM CaCl₂) and deionized water, respectively. They were photographed before the shoot length were measured. The seedling samples were dried at 70° C. in an oven for 7 d and the dry weight of the seedlings was measured. The experiments were conducted with three biological replicates.

Plantation of Rice in Soils Treated with as and/or Cd

The control soil used in this study contained background levels of As at 2.09 mg/kg and Cd at 0.44 mg/kg. The control soil was supplemented with 10 mg/kg As in the form of NaAsO₂ and/or 3 mg/kg Cd in the form of CdSO₄. Rice seedlings were grown in control soil in nursey for 28 d. There were then transplanted onto soils with or without As and/or Cd treatment and grown to maturity in the greenhouse. Rice seeds and straw were harvested and dried for further analysis. The experiments were conducted with three biological replicates.

Isolation of Intact Protoplasts and Vacuoles from Rice Mesophyll Cells and Treatment of Protoplasts with CdSO₄ or PC2-Cd Complex

The protoplasts were isolated from rice mesophyll cells as described previously (Trinidad et al., 2021). In brief, the shoots from 10-day-old seedlings of rice germinated and grown on half-strength MS medium were cut into 0.5 cm strips. The protoplasts were released from the strips by adding protoplast isolation buffer [0.6 mannitol, 10 mM methyl ethane sulfonate (MES), 10 mM CaCl₂), 0.1% BSA (w/v), 1.5% (w/v) cellulase RS (C0615, Sigma, USA), and 0.75% (w/V) pectinase RS (P2401, Sigma, USA)] followed by incubation in the dark with gentle shaking for 4 h at 28° C. The protoplasts were collected by centrifugation at 150 g for 5 min at 20° C. using a swinging bucket rotor with slow acceleration and slow deceleration setting. The pellet was washed twice with 20 ml W5 buffer (154 mM NaCl, 125 mM CaCl₂, 5 mM KCl and 2 mM MES) and re-collected by spinning at 100 g for 3 min. To isolate the intact vacuoles, 3 ml lysis buffer [0.2M mannitol, 10% Ficoll-400, 15 mM EDTA (pH 8.0), 5 mM sodium phosphate (pH 8.0)] pre-warmed 37° C. was added to the protoplasts. The protoplasts were resuspended gently by being pipetted up and down for 5-8 times and lysed by incubation in a warm water bath at 37° C. for 5-10 min. The vacuoles released from protoplasts were purified by centrifugation on a three-step Ficoll®-400 gradient. One volume of lysed protoplast suspension was overlaid with two volumes of Ficoll®-400 solution (5% in w/v), prepared by mixing one volume of lysis buffer and one volume vacuole buffer (30 mM KCl, 20 mM HEPES-KOH, pH7.5, 0.4 M betaine, 15 mg mL⁻¹ BSA, and 1 mM DDT). One volume vacuole buffer was then layered on the top of the gradient carefully but quickly. The vacuoles were collected on the interface between 5% Ficoll®-400 solution and vacuole buffer after centrifugation at 1,500 g for 20 min. The PC₂—Cd complex was formed by mixing CdSO₄, PC₂ and DDT at a molar ratio of 1:1:1 and incubating at 25° C. for 1 h. The protoplasts were resuspended in MMG buffer (0.4 M mannitol, 15 mM MgCl₂, and 4 mM MES, adjust pH to 7.5 with KOH). 10 μM CdSO4 or PC₂—Cd complex prepared above was added to protoplasts in MMG buffer and then incubated in the dark at room temperature for 1 h. After incubation, the protoplasts were collected by centrifugation at 150 g for 5 min at 20° C. The protoplasts were washed three times with W5 buffer to remove residual Cd in the buffer. Protoplasts and vacuoles isolated from the protoplasts were used for Cd determination by ICP-MS.

### Element Analysis by Inductively Coupled Plasma Mass Spectrometry (ICP-MS)

The concentration of elements in brown rice (de-husked but unpolished rice seeds) and straw was determined by ICP-MS. About 0.1 g dried rice seeds or straw tissues were pre-digested with 3 ml concentrated HNO₃/H₂O₂ mixture (5:1, v:v) overnight at room temperature, and were then digested in a microwave oven (Ethos One, Milestone Technologies). The elements concentration in the digested solution was determined by ICP-MS (7700S, Agilent Technologies, USA) after dilution. The rice flour NIST® SRM® 1568b was used for certified reference material (CRM) to assess the precision and accuracy of analysis procedures.

### Statistical Analysis

Data were analysed using two-tailed Student's t test (* P<0.05 or ** P<0.01) or one-way ANOVA followed by LSD's test (significance level of P<0.05). All analysis was performed using IBM SPSS statistics 19 software.

### Example 2

### Generation of OsHMA3, OsABCC1 and OsPCS1-Overexpressed Lines

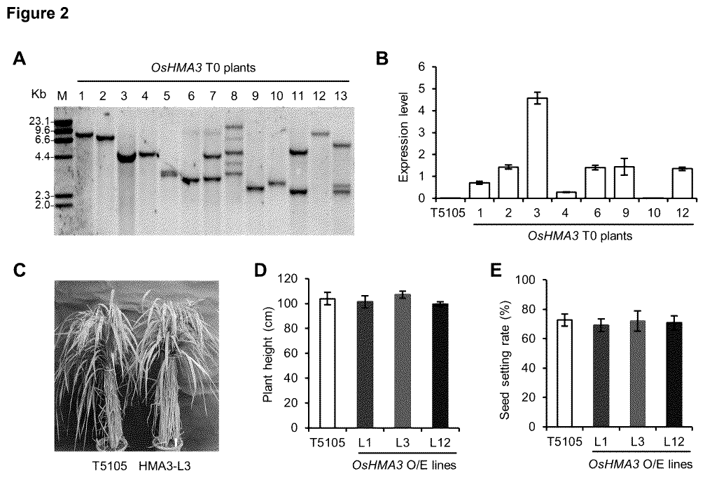

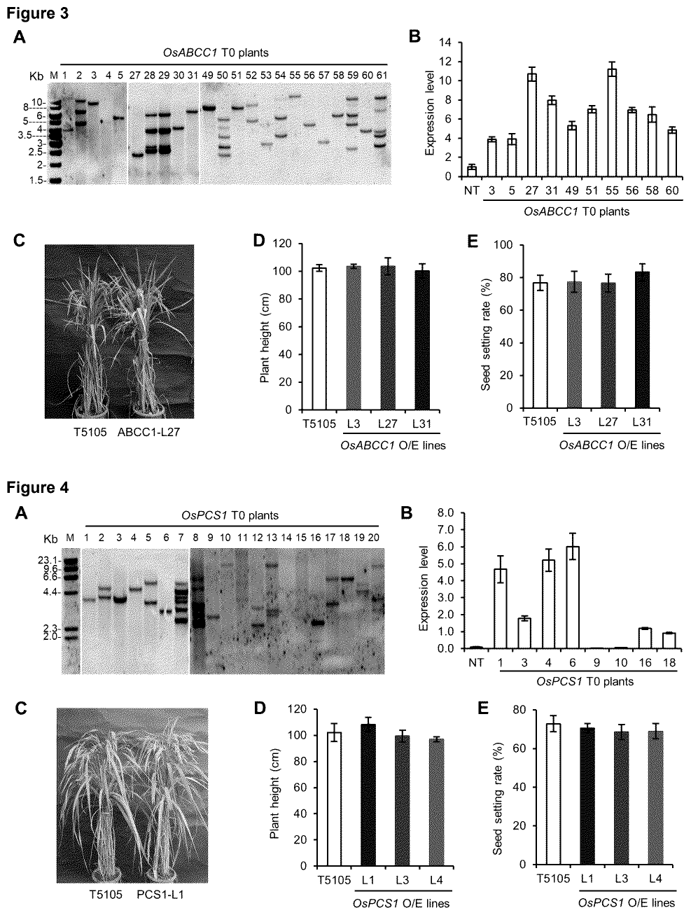

Three binary constructs containing the coding regions of cDNA or genomic clones of OsHMA3, OsPCS1 and OsABCC1 genes under the control of OsActin1 promoter were made and used to generate transgenic rice plants in T5105 genetic background via Agrobacterium-mediated rice transformation (FIG. 1A to 1D). Transgenic TO plants were characterized to screen for transgenic lines that carried a single-copy of T-DNA and showed over expression of the transgenes for further study (FIGS. 2A and 2B, FIGS. 3A and 3B, FIGS. 4A and 4B, FIGS. 5A and 5B, FIGS. 6A and 6B, FIGS. 8A and 8B, Table 2). qPCR and Southern blotting methods were used to screen for typical lines. Primers and probes used are listed in Table 1. Three independent transgenic lines for each gene were selected and characterized in details for growth and development, yield and gene expression. All these transgenic lines displayed similar morphological phenotypes in plant height and panicle as well as seed setting rate to those of the non-transgenic control T5105 (FIGS. 2C to 2E, FIGS. 3C to 3E; FIGS. 4C to 4E).

TABLE 2
Summary of transgenic plants generated in this study

| | | No. of T0 plants with |
|---|---|---|
| | No. of | single-copy T-DNA and |
| Gene of interest | T0 plants | overexpression of transgenes |
| P_{Actin1}:gABCC1:T_{Nos} | 62 | 10 |
| P_{Actin1}:cHMA3:T_{Nos} | 22 | 7 |
| P_{Actin1}:cPCS1:T_{Nos} | 27 | 6 |

### Example 3

### The Cd Concentration in Grains of the OsHMA3-Overexpressed Lines was Significantly Decreased

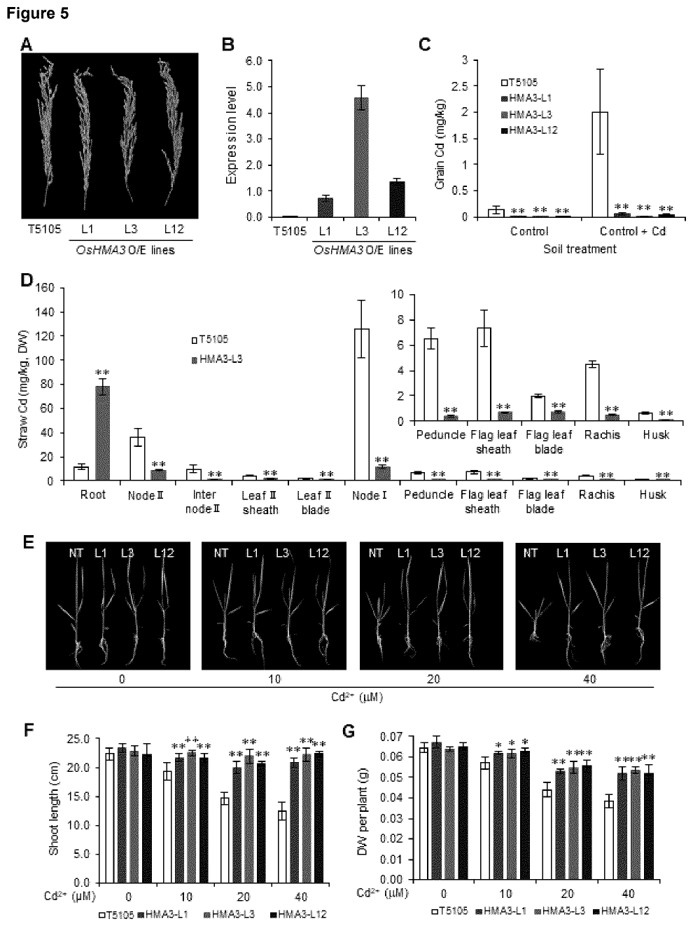

T5105 and the OsHMA3-overexpressed lines (HMA3-L1, HMA3-L3 and HMA3-L12) were grown in control soil and soil containing 3 mg/kg Cd in the form of CdSO₄. After harvest, the seeds and straw were subjected to ICP-MS analysis. The grain Cd concentrations of the OsHMA3-overexpressed lines in the control soil (0.003±0.001 mg/kg) and the Cd-treated soil (0.041±0.012 mg/kg) was only 2.0% and 2.0%, respectively, to those of T5105 in the control experiments (0.135±0.064 mg/kg and 2.010±0.813 mg/kg), respectively (FIG. 5C). In the straw of T5105 grown in Cd-treated soil, the stem nodes (Node I and Node II) had relatively higher levels of Cd concentration than roots, internodes, leaves (leaf sheath and blade) and panicles (peduncle, rachis and husk) (FIG. 5D). In the straw of HMA3-L3 grown in the Cd-treated soil, roots had the highest level of Cd concentration than other straw tissues (FIG. 5D). The Cd concentration in the roots of HMA3-L3 (78.001±7.056 mg/kg) was 6.7-fold higher than that in the roots of T5105 (11.697±2.408 mg/kg), whereas the Cd concentrations in Node I (11.704±1.447 mg/kg) and Node II (9.002±0.590 mg/kg) of HMA3-L3 were 9.3% and 25.0%, respectively, to those in Node I (125.837±24.001 mg/kg) and Node II (36.006±7.557 mg/kg) of T5105 (FIG. 5D). These results demonstrated that the expression of the P_{Actin1}:cHAM3:T_{Nos} gene in the transgenic plants significantly enhanced Cd accumulation in roots and greatly decreased Cd allocation to aerial parts of plants, including seeds.

Seedlings of T5105 and OsHMA3-overexpressed lines were tested on a half-strength MS medium for tolerance to Cd. T5105 plants displayed increased growth retardation to Cd treatment at the concentrations from 10 to 40 μM CdSO₄ (FIG. 5E). Compared with the untreated T5105 plants, both shoot length and dry weight of the treated T5105 plants were reduced at 14 d after treatment (FIGS. 5F and 5G). Compared with the untreated T5105 and transgenic plants, the OsHMA3-overexpressed lines grown in Cd-containing medium did not show obvious growth retardation at 14 d after Cd treatment (FIGS. 5E and 5F). The dry weight of the OsHMA3-overexpressed lines was slightly reduced than the untreated rice seedlings (FIG. 5G). But the reduction was much lower than that of T5105 after treatment with Cd (FIG. 5G). These results indicate that overexpression of OsHMA3 confer great tolerance to Cd.

### Example 4

### The as Concentration in the Rice Grains of the OsABCC1-Overexpressed Lines was Partially Reduced

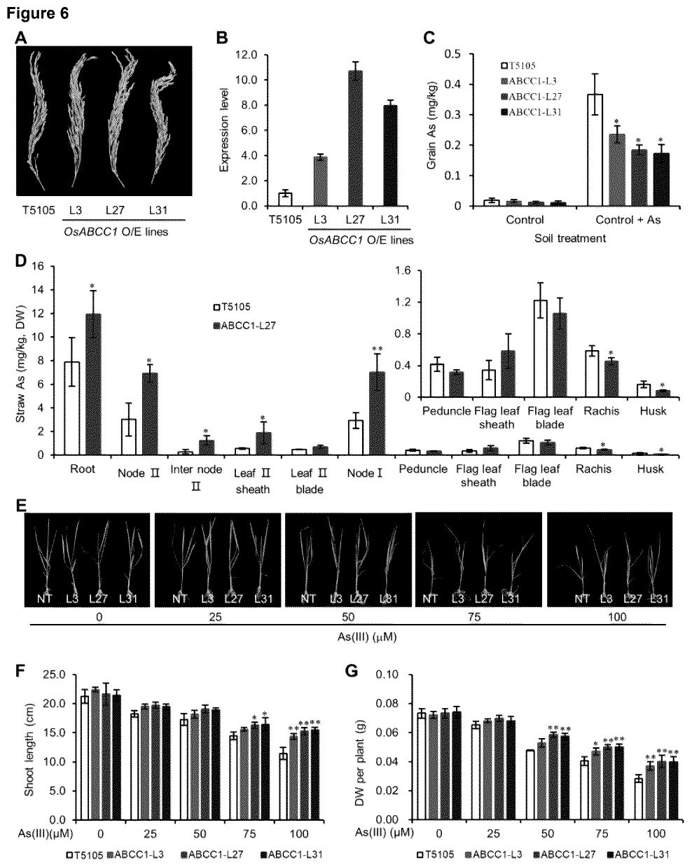

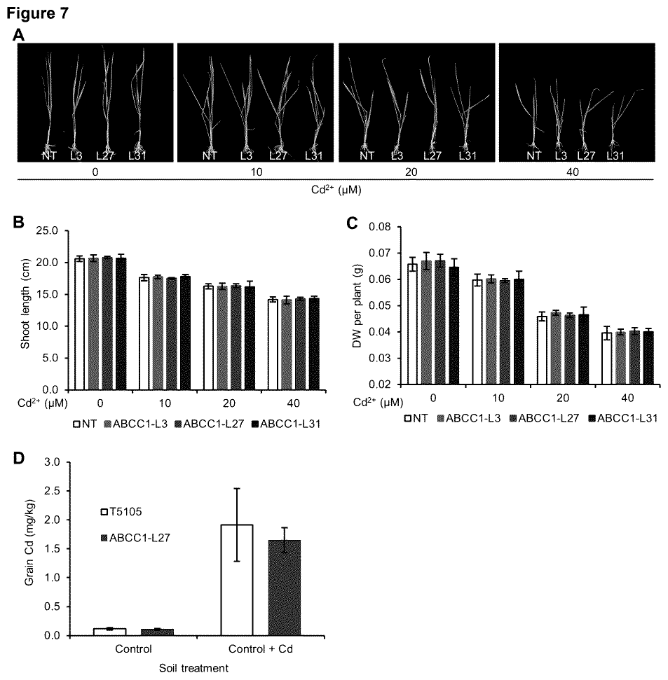

The As concentration in seeds and straw of T5105 and the independent OsABCC1-overexpressed lines grown in control soil and soil containing 10 mg/kg As in the form of NaAsO₂ were determined by ICP-MS (FIGS. 6C and 6D). The mean grain As concentrations of the OsABCC1-overexpressed lines grown in the control soil (0.013±0.005 mg/kg) and the As-treated soil (0.197±0.024 mg/kg) were 72.2% and 53.7% to those of T5105 in the control experiments (0.018±0.008 mg/kg and 0.367±0.068 mg/kg), respectively (FIG. 6C). For plants grown in the As-treated soil, the As concentrations in roots, stems (Node I, Node II, Internode II) and lower leaves of ABCC1-L27 were higher than those of T5105, whereas the As concentrations in flag leaf blades and panicles (peduncle, rachis and husk) of ABCC1-L27 was similar to or lower than those of T5105 (FIG. 6D). Seedlings of T5105 and the OsABCC1-overexpressed lines were tested on a half-strength MS medium for tolerance to As. No significant difference in shoot length and dry weight per plant was observed between T5105 and the OsABCC1-overexpressed lines when they were treated with 25 μM As (III) in the form of NaAsO₂ (FIGS. 6E to 6G). However, the OsABCC1-overexpressed lines displayed enhanced tolerance to 50-100 μM As (III) with longer shoot length and higher dry weight than those of T5105 (FIGS. 6E to 6G). The OsABCC1-overexpressed lines were also tested for Cd tolerance at the seedling stage. Compared to T5105, they did not show any enhanced tolerance to Cd treatment (FIGS. 7A to 7C). Further investigation also demonstrated that the grains harvested from ABCC1-L27 grown on control or Cd-treated soils had similar levels of Cd concentration to that of T5105 (FIG. 7D). The results indicated that the expression of the P_{Actin1}:gABCC1:T_{Nos} gene in the transgenic plants enhanced the accumulation As in roots, stems (Node I, Node II, Internode II) and lower leaves and decreased the As concentration in grains.

### Example 5

The as and Cd Concentrations in the Grains of the OsPCS1-Overexpressed Lines were Partially Reduced

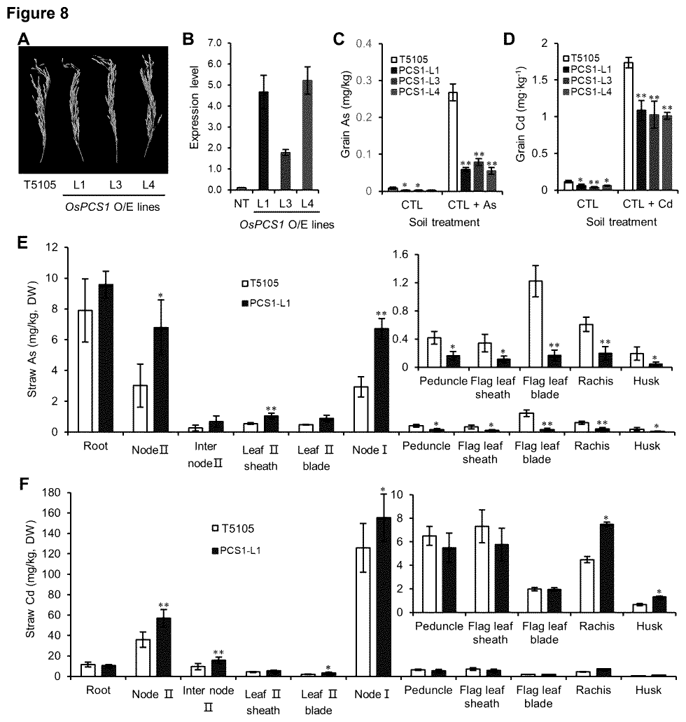

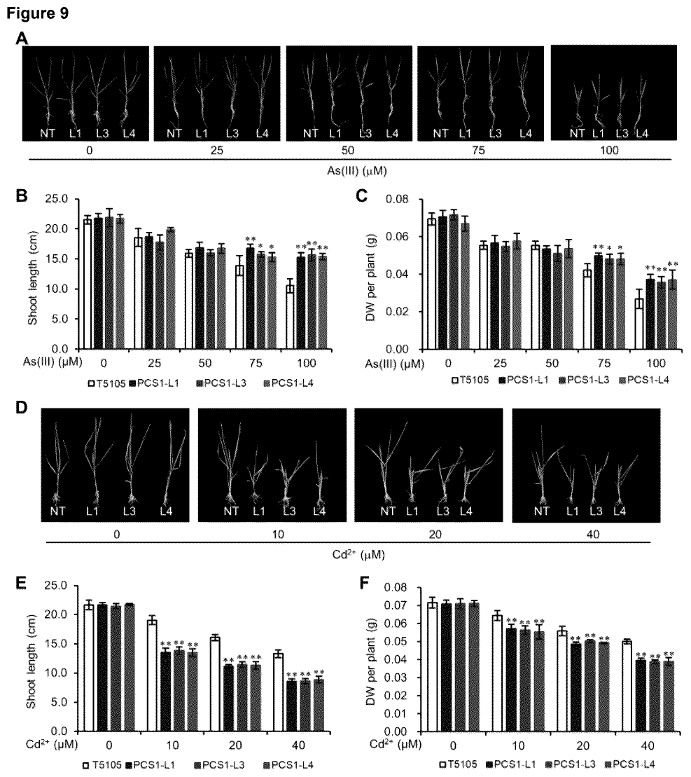

The overexpression of the OsPCS1 gene in T5105 caused a significant reduction in As and Cd concentrations in rice grain (FIGS. 8C and 8D). For rice plants grown in the control soil, the mean grain As concentration of the OsPCS1-overexpressed lines (0.003±0.001 mg/kg) was 37.5% to that of T5105 (0.008±0.003 mg/kg) (FIG. 8C). For rice plants grown in the As-treated soil, the mean grain As concentration of the OsPCS1-overexpressed lines (0.064±0.008 mg/kg) was 23.9% to that of T5105 (0.268±0.023 mg/kg) (FIG. 8C). In the plantation experiments with Cd treatment, the mean grain Cd concentrations of the OsPCS1-overexpressed lines grown in the control soil (0.053±0.011 mg/kg) and the Cd-treated soil (1.042±0.122 mg/kg) were 46.1% and 60.0% to those of T5105 in the control experiments (0.115±0.018 mg/kg and 1.736±0.070 mg/kg), respectively (FIG. 8D). It was observed that the reduction on grain As concentration was more significant than that on grain Cd concentration in the OsPCS1-overexpressed lines (FIG. 8C). The As and Cd concentrations in different parts of rice straw were measured as well. When grown in As-treated soil, the OsPCS1-overexpressed line PCS1-L1 had higher levels of As concentration in roots, nodes (Node I and Node II), internode II and leaf II, but lower levels of As concentration in peduncle, flag leaf blade, rachis and husk than T5105 (FIG. 8E). When grown in the Cd-treated soil, PCS1-L1 had higher levels of Cd concentration in nodes (Node I and Node II), inter node II and rachis, but only had similar or even lower levels of Cd concentration in roots, flag leaves and peduncle of straw than T5105 (FIG. 8F). Unlike the highest As concentration was detected in roots of T5105 and PCS1-L1, the Cd concentration in the roots of the two lines was much lower than that in the nodes (FIGS. 8E and 8F). In the seedling tests for tolerance to As or Cd, the OsPCS1-overexpressed lines showed slightly enhanced tolerance to 75-100 μM As (III) in the form of NaAsO₂ with longer shoot height and higher dry weight than those of T5105 (FIGS. 9A to 9C). However, the OsPCS1-overexpressed lines displayed significant hypersensitivity to 10-40 μM Cd²⁺ with shorter shoot length and lower dry weight than those of T5105 (FIGS. 9D to 9F). The results demonstrated that the expression of the P_{Actin1}:cPCS1:T_{Nos} gene in the OsPCS1-overexpressed lines enhanced As accumulation in the roots, stems and lower leaves and decreased As allocation to flag leaves, panicles and seeds. In the meanwhile, the expression of the P_{Actin1}:cPCS1:T_{Nos} gene in the OsPCS1-overexpressed lines enhanced Cd accumulation mainly in stems of the transgenic plants.

### Example 6

Co-Expression of the P_{Actin1}:gABCC1:T_{Nos} gene and the P_{Actin1}:cPCS1:T_{Nos} Gene Showed Synergistic Effect on Reducing as Concentration in Rice Grain

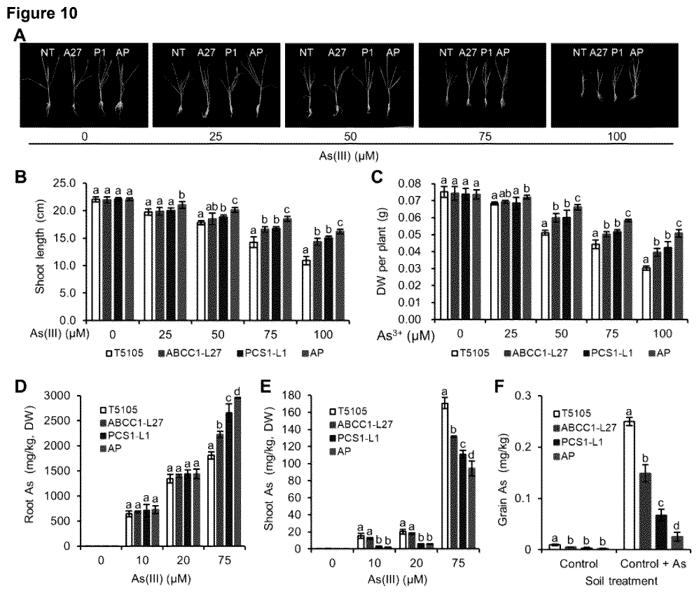

In rice plants, As (III) was sequestrated into vacuoles in the form of phytochelatin-arsenic (PC—As) complexes through ABCC1 (Hayashi et al., 2017; Song et al., 2014). To further investigate if the co-overexpression of OsABCC1 and OsPCS1 had synergistic effect on decreasing As concentration in rice grain, a AP line was generated to pyramid the P_{Actin1}:gABCC1:T_{Nos} gene and the P_{Actin1}:cPCS1:T_{Nos} gene into a single line by crossing ABCC1-L27 with PCS1-L1 followed by self-pollination and selection of double homozygotes. Indeed, compared to ABCC1-L27 or PCS1-L1, AP showed an synergistically enhanced tolerance to 25-100 μM As (III) at the seedling stages (FIGS. 10A to 10C). In addition, AP had a higher As concentration in roots and a lower As concentration in shoots than those of ABCC1-L27 or PCS1-L1, especially in the treatment with 75 μM As (III) at the seedling stages (FIGS. 10D and 10E). The As concentration in the grains of AP grown in As-treated soil (0.025±0.009 mg/kg) was much lower than that of ABCC1-L27 (0.149±0.000 mg/kg) or PCS1-L1 (0.067±0.002 mg/kg), and was only 10.0% to that of T5105 (0.250±0.008 mg/kg) (FIG. 10F). The results demonstrated that the co-expression of the P_{Actin1}:gABCC1:T_{Nos} gene and the P_{Actin1}:cPCS1:T_{Nos} gene in transgenic rice plants showed synergistic effect on reducing As concentration in rice grain and providing enhanced tolerance to As treatment at the seedling stages.

### Example 7

### The Hypersensitivity of the OsPCS1-Overexpressed Lines to Cd Treatment was Relieved by the Co-Overexpression of the OsHMA3 Gene

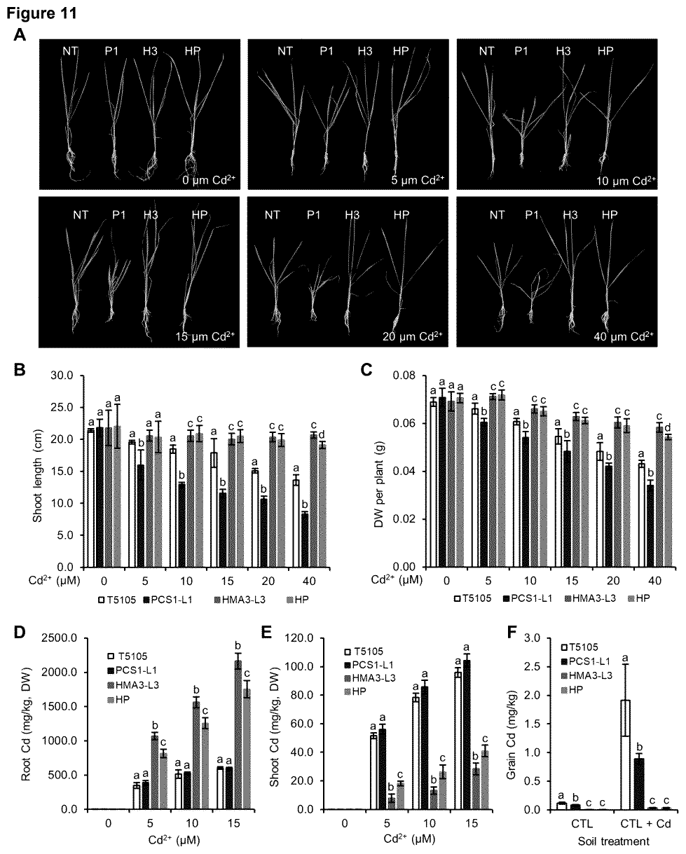

To investigate if the co-overexpression of the OsHMA3 gene can relieve the hypersensitivity of the OsPCS1-overexpressed lines to Cd treatment, a transgenic line (HP) carrying both the P_{Actin1}:cPCS1:T_{Nos} gene and the P_{Actin1}:cHMA3:T_{Nos} gene was generated by crossing PCS1-L1 and HMA3-L3 followed by self-pollination and selection of double homozygotes. Seedlings of HP, PCS1-L1, HMA3-L3 and T5105 were tested on Cd²⁺-containing half-strength MS medium. The HP line provided a similar level of enhanced tolerance to Cd treatment to HMA3-L3 with longer shoot length and higher dry weight than those of PCS1-L1 or T5105 (FIGS. 11A to 11C). ICP-MS analysis with the tissues from the treated seedlings indicated that the HP plants had more Cd accumulated in roots and less Cd distributed to shoots than those of the PCS1-L1 or T5105 plants, which was similar to the pattern of HMA3-L3 plants (FIGS. 11D and 11E). The mean Cd concentrations in the grains of the HP plants grown in the control soil (0.004±0.001 mg/kg) and the Cd-treated soil (0.032±0.011 mg/kg) were similar to those of the HMA3-L3 plants (0.004±0.001 mg/kg and 0.036±0.006 mg/kg), but significantly lower than those of PCS1-L1 (0.085±0.008 mg/kg and 0.889±0.093 mg/kg) and T5105 (0.115±0.019 mg/kg and 1.913±0.630 mg/kg) in the control experiments (FIG. 11F).

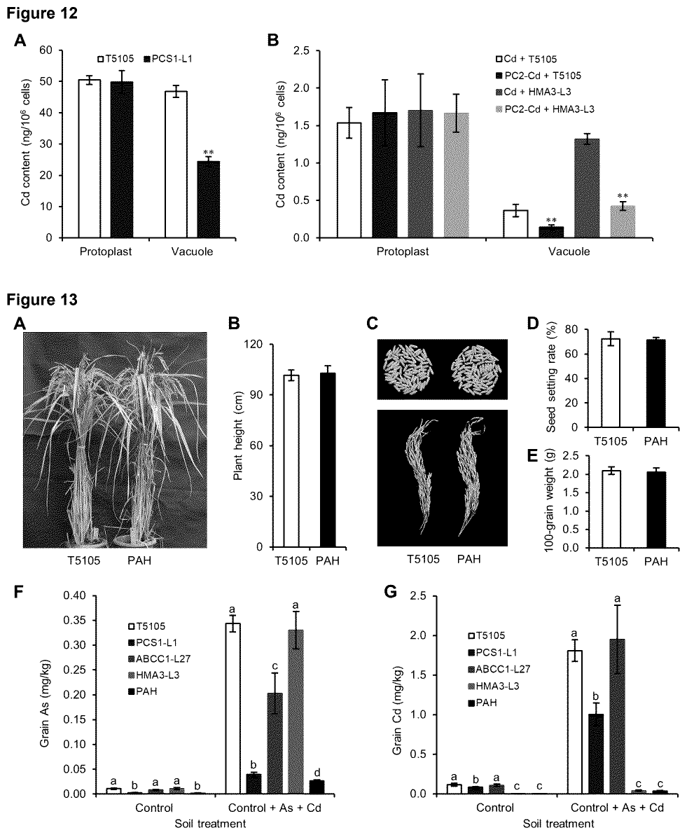

To further investigate the effect of OsPCS1 overexpression on Cd sequestration in vacuoles of rice cells, protoplasts and vacuoles were isolated from 10-day-old shoots of T5105 and PCS1-L1 seedlings that were grown on Cd-containing medium and subjected to Cd concentration measurement by ICP-MS analysis. There was no significant difference in the Cd concentration in the protoplasts of T5105 (50.468±1.410 ng/10⁶ cells) and PCS1-L1 (49.839±3.694 ng/10⁶ cells) (FIG. 12A). However, the Cd concentration in the vacuoles of PCS1-L1 (24.429±1.498 ng/10⁶ cells) was only 52.2% to that of T5105 (46.809±1.933 ng/10⁶ cells) (FIG. 12A). The results indicated that the expression of the P_{Actin1}:cPCS1:T_{Nos} gene in PCS1-L1 suppressed the sequestration of Cd to vacuoles. The overexpression of the OsPCS1 gene in rice would increase the synthesis of PCs and more PC—Cd complex would be formed in cytosol. The next step of the study was to investigate if the PC2-Cd complex would affect Cd sequestration to vacuoles through HMA3. Protoplasts isolated from T5105 and HMA3-L3 were incubated with Cd²⁺ and PC2-Cd complex, respectively. The Cd concentration in the incubated protoplasts and their vacuoles were then determined. A similar level of Cd concentration was detected in all of the protoplast samples, indicating that both Cd and PC2-Cd could be transported into protoplasts of T5105 or HMA3-L3 at similar efficiency (FIG. 12B). For protoplasts incubated with Cd²⁺, the Cd concentration in the vacuoles of HMA3-L3 (1.318±0.073 ng/10⁶ cells) were 3.6-fold higher than that in the vacuoles of T5105 (0.366±0.081 ng/10⁶ cells), confirming that overexpression of OsHMA3 enhanced Cd²⁺ sequestration to vacuoles (FIG. 12B). For protoplasts incubated with PC₂—Cd, the Cd concentrations in the vacuoles of T5105 (0.141±0.029 ng/10⁶ cells) and HMA3-L3 (0.423±0.058 ng/10⁶ cells) were 38.5% and 32.1%, respectively, to those in the vacuoles of the T5105 and HMA3-L3 protoplasts incubated with Cd²⁺ (FIG. 12B). In the meanwhile, the Cd concentration in the vacuoles of HMA3-L3 incubated with PC₂—Cd (0.423±0.058 ng/10⁶ cells) was 3-fold higher than that in the vacuoles of T5105 in the control experiment (0.141±0.029 ng/10⁶ cells) (FIG. 12B). The results suggested that PC2-Cd complex suppressed HMA3-mediated sequestration of Cd²⁺ to vacuoles and the overexpression of the OsHMA3 gene could relieve this suppression caused by over-produced PC₂ or other PCs due to the overexpression of the OsPCS1 gene in rice cells.

### Example 8

Generation of Low-As and Low-Cd Rice Grains by Pyramiding P_{Actin1}:cHMA3:T_{Nos}, P_{Actin1}:cPCS1:T_{Nos} and P_{Actin1}:gABCC1:T_{Nos} in a Single Rice Line

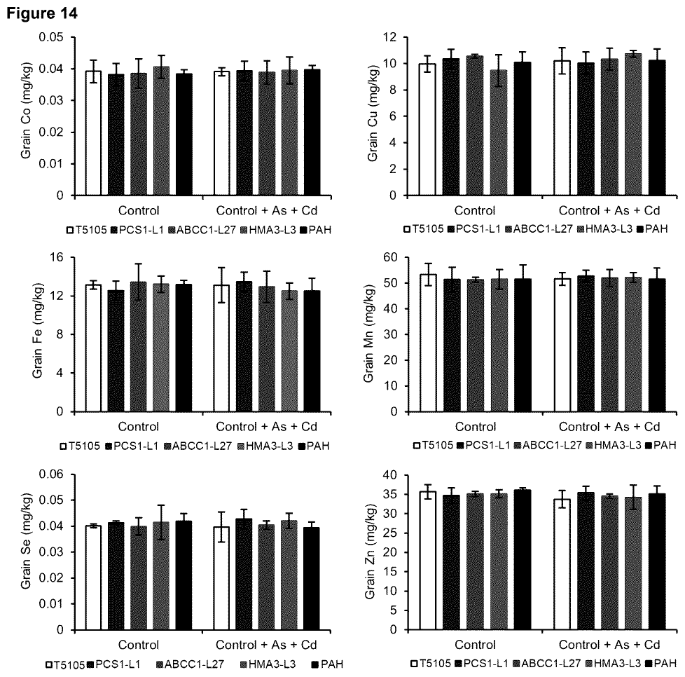

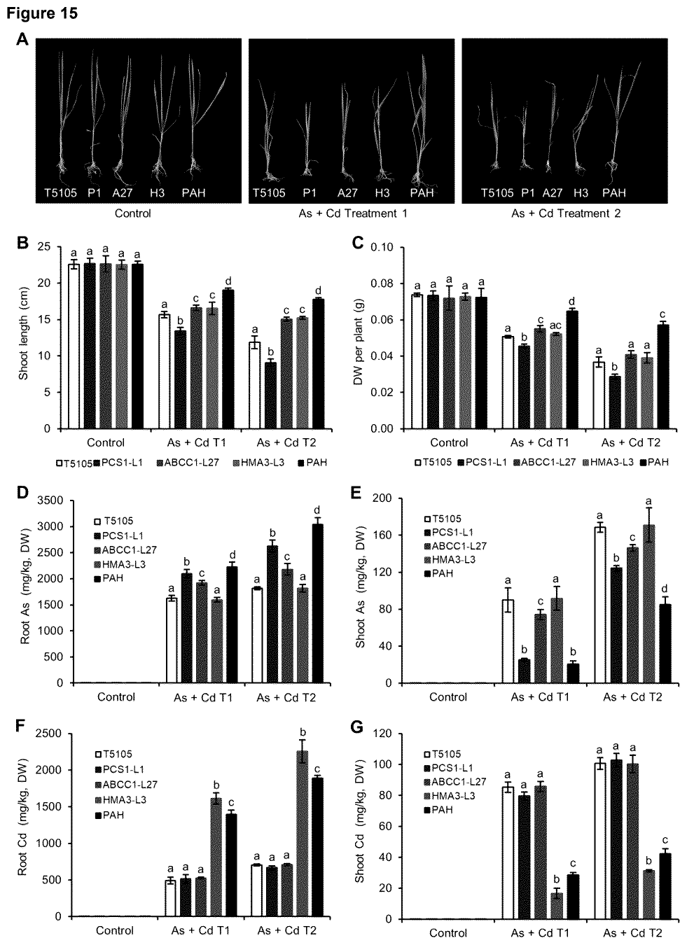

To generate rice grains with low concentration for both As and Cd elements, a rice line (PAH) with the co-overexpression of the OsPCS1, OsABCC1 and OsHMA3 genes was developed through crossing and marker assisted selection with the P_{Actin1}:cPCS1:T_{Nos} gene from PCS1-L1, the P_{Actin1}:gABCC1:T_{Nos} gene from ABCC1-L27 and the P_{Actin1}:cHMA3:T_{Nos} gene from HMA3-L3. Like its parental transgenic lines, the PAH line showed normal growth and development with similar plant architecture, growth duration, panicles and seeds, seed-setting rate and 100-grain weight to those of T5105 (FIGS. 13A to 13E). Plants of PAH, PCS1-L1, ABCC1-L27, HMA3-L3 and T5105 were grown on control soil and soil treated with both As and Cd, respectively, and the grains were subjected to As and Cd concentration measurement by ICP-MS analysis. For the plants grown on the control soil, the grain As concentration of PAH (0.002±0.000 mg/kg) was 18.2% to that of T5105 (0.011±0.001 mg/kg), whereas the grain Cd concentration of PAH (0.004±0.001 mg/kg) was 3.5% to that of T5105 (0.115±0.019 mg/kg) (FIGS. 13F and 13G). For the plants grown on the As- and Cd-treated soil, the grain As concentration of PAH (0.027±0.002 mg/kg) was 7.9% to that of T5105 (0.343±0.017 mg/kg), whereas the grain Cd concentration of PAH (0.036±0.006 mg/kg) was 2.0% to that of T5105 (1.809±0.136 mg/kg) (FIGS. 13F and 13G). In the experiments with both kinds of soils, the As or Cd concentration in the grains of PAH was either lower or comparable to those in the grains of parental lines carrying single transgenes (FIGS. 13F and 13G). It should be noted that, compared to T5105, the over-expression of the OsHMA3 gene in HMA3-L3 did not make any significant change to grain As concentration, while the over-expression of OsABCC1 gene in ABCC1-L27 did not affect grain Cd concentration (FIGS. 13F and 13G). The results suggest that the PCs-dependent and OsABCC1-mediated As sequestration to vacuoles and the OsHMA3-mediated Cd sequestration to vacuoles are independent to each other. ICP-MS analysis also indicated that the concentrations of the micronutrient elements, including cobalt (Co), cupper (Cu), iron (Fe), manganese (Mn), selenium (Se) and zinc (Zn), in the grains of the 4 lines grown on the control soils or As- and Cd-treated soils did not show any significant difference (FIG. 14). Seedlings of the transgenic lines and T5105 were tested on half-strength MS medium containing 50 μM As (III) and 10 μM Cd²⁺ (Treatment 1) or 75 μM As (III)+20 μM Cd²⁺ (Treatment 2). In both treatments, the PAH seedlings displayed the highest enhanced tolerance to As and Cd double treatment among all of the lines tested with the longest shoot length and the greatest dry weight (FIGS. 15A to 15C). Compared to T5105, the PAH seedlings accumulated much higher As or Cd concentration in roots and lower As or Cd concentration in shoots (FIGS. 15D to 15G). The results collectively demonstrated that the co-expression of the P_{Actin1}:cPCS1:T_{Nos} gene, P_{Actin1}:gABCC1:T_{Nos} gene and the P_{Actin1}:cHMA3:T_{Nos} gene in the PAH line reduced As and Cd concentrations significantly in rice grain and provided enhanced tolerance to As and Cd double treatment at the seedling stages by increasing the accumulation and sequestration of the two toxic elements in roots and decreasing the transport of As and Cd to the aerial parts of tissues and organs, especially the seeds, without any trade-off in plant growth and development, yield and micronutrients.

In summary, a genetic engineering approach was used to generate transgenic plants that overexpress OsABCC1, OsPCS1 and OsHMA3 under the OsActin1 promoter in the T5105 genetic background. The expression of the P_{Actin1}:cPCS1:T_{Nos} gene or P_{Actin1}:gABCC1:T_{Nos} gene alone partially reduced As concentration in the grains of transgenic rice plants. The co-expression of the P_{Actin1}:cPCS1:T_{Nos} gene and P_{Actin1}:gABCC1:T_{Nos} gene significantly reduced As concentration in the grains of the double genes over-expressed transgenic plants. The expression of the P_{Actin1}:cHMA3:T_{Nos} gene significantly reduced Cd concentration in the grains of transgenic rice lines. The co-expression of the P_{Actin1}:cPCS1:T_{Nos} gene, the P_{Actin1}:gABCC1:T_{Nos} gene and the P_{Actin1}:cHMA3:T_{Nos} gene significantly reduced both As and Cd concentration in the grains of triple genes over-expressed transgenic plants. All transgenic plants showed similar normal growth and development to the non-transgenic T5105 without any pleiotropic phenotype or yield penalty. The low-As and low-Cd rice will reduce the uptake of two toxic elements by human beings through the consumption of rice and benefit our health.

### BIBLIOGRAPHY

1. Das, N., Bhattacharya, S., Bhattacharyya, S., and Maiti, M. K. (2017). Identification of alternatively spliced transcripts of rice phytochelatin synthase 2 gene OsPCS2 involved in mitigation of cadmium and arsenic stresses. Plant molecular biology 94, 167-183.
2. Deng, F., Yamaji, N., Ma, J. F., Lee, S. K., Jeon, J. S., Martinoia, E., Lee, Y., and Song, W. Y. (2018). Engineering rice with lower grain arsenic. Plant Biotechnol J 16, 1691-1699.
3. Fowler, B. A. (2009). Monitoring of human populations for early markers of cadmium toxicity:

a review. Toxicology and applied pharmacology 238, 294-300.
4. Grill, E., Winnacker, E.-L., and Zenk, M. H. (1985). Phytochelatins: the principal heavy-metal complexing peptides of higher plants. Science 230, 674-676.
5. Ha, S. B., Smith, A. P., Howden, R., Dietrich, W. M., Bugg, S., O'Connell, M. J., Goldsbrough, P. B., and Cobbett, C. S. (1999). Phytochelatin synthase genes from Arabidopsis and the yeast Schizosaccharomyces pombe. Plant Cell 11, 1153-1164.
6. Hayashi, S., Kuramata, M., Abe, T., Takagi, H., Ozawa, K., and Ishikawa, S. (2017). Phytochelatin synthase OsPCS1 plays a crucial role in reducing arsenic levels in rice grains. The Plant journal: for cell and molecular biology 91, 840-848.
7. Hiei, Y., Ohta, S., Komari, T., and Kumashiro, T. (1994). Efficient transformation of rice (Oryza sativa L.) mediated by Agrobacterium and sequence analysis of the boundaries of the T-DNA. The Plant journal: for cell and molecular biology 6, 271-282.
8. Hughes, M. F. (2002). Arsenic toxicity and potential mechanisms of action. Toxicology letters 133, 1-16.
9. Li, J. C., Guo, J. B., Xu, W. Z., and Ma, M. (2007). RNA interference-mediated silencing of phytochelatin synthase gene reduce cadmium accumulation in rice seeds. Journal of integrative plant biology 49, 1032-1037.
10. Luo, Y., and Yin, Z. (2013). Marker-assisted breeding of Thai fragrance rice for semi-dwarf phenotype, submergence tolerance and disease resistance to rice blast and bacterial blight. Molecular breeding 32, 709-721.
11. Ma, J. F., Yamaji, N., Mitani, N., Xu, X. Y., Su, Y. H., McGrath, S. P., and Zhao, F. J. (2008). Transporters of arsenite in rice and their role in arsenic accumulation in rice grain. Proceedings of the National Academy of Sciences of the United States of America 105, 9931-9935.
12. Miyadate, H., Adachi, S., Hiraizumi, A., Tezuka, K., Nakazawa, N., Kawamoto, T., Katou, K., Kodama, I., Sakurai, K., Takahashi, H., et al. (2011). OsHMA3, a P1B-type of ATPase affects root-to-shoot cadmium translocation in rice by mediating efflux into vacuoles. The New phytologist 189, 190-199.
13. Panda, S. K., Upadhyay, R. K., and Nath, S. (2010). Arsenic Stress in Plants. Journal of Agronomy and Crop Science 196, 161-174.
14. Park, S.-H., Yi, N., Kim, Y. S., Jeong, M.-H., Bang, S.-W., Choi, Y. D., and Kim, J.-K. (2010). Analysis of five novel putative constitutive gene promoters in transgenic rice plants. Journal of Experimental Botany 61, 2459-2467.
15. Reece, K. S., McElroy, D., and Wu, R. (1990). Genomic nucleotide sequence of four rice (Oryza sativa) actin genes. Plant molecular biology 14, 621-624.
16. Shao, J. F., Xia, J., Yamaji, N., Shen, R. F., and Ma, J. F. (2018). Effective reduction of cadmium accumulation in rice grain by expressing OsHMA3 under the control of the OsHMA2 promoter. Journal of Experimental Botany 69, 2743-2752.
17. Song, W. Y., Yamaki, T., Yamaji, N., Ko, D., Jung, K. H., Fujii-Kashino, M., An, G., Martinoia, E., Lee, Y., and Ma, J. F. (2014). A rice ABC transporter, OsABCC1, reduces arsenic accumulation in the grain. Proceedings of the National Academy of Sciences of the United States of America 111, 15699-15704.
18. Song, Y., Wang, Y., Mao, W., Sui, H., Yong, L., Yang, D., Jiang, D., Zhang, L., and Gong, Y. (2017). Dietary cadmium exposure assessment among the Chinese population. PLOS One 12, e0177978.
19. Sui, F.-Q., Chang, J.-D., Tang, Z., Liu, W.-J., Huang, X.-Y., and Zhao, F.-J. (2018). Nramp5 expression and functionality likely explain higher cadmium uptake in rice than in wheat and maize. Plant and Soil 433, 377-389.
20. Takahashi, R., Ishimaru, Y., Shimo, H., Ogo, Y., Senoura, T., Nishizawa, N. K., and Nakanishi, H. (2012). The OsHMA2 transporter is involved in root-to-shoot translocation of Zn and Cd in rice. Plant, cell & environment 35, 1948-1957.
21. Trinidad, J. L., Longkumer, T., and Kohli, A. (2021). Rice Protoplast Isolation and Transfection for Transient Gene Expression Analysis. Methods in molecular biology 2238, 313-324.
22. Tsukahara, T., Ezaki, T., Moriguchi, J., Furuki, K., Shimbo, S., Matsuda-Inoguchi, N., and Ikeda, M. (2003). Rice as the most influential source of cadmium intake among general Japanese population. Science of The Total Environment 305, 41-51.
23. Ueno, D., Yamaji, N., Kono, I., Huang, C. F., Ando, T., Yano, M., and Ma, J. F. (2010). Gene limiting cadmium accumulation in rice. Proceedings of the National Academy of Sciences of the United States of America 107, 16500-16505.
24. Wang, F., Wang, Z., and Zhu, C. (2012). Heteroexpression of the wheat phytochelatin synthase gene (TaPCS1) in rice enhances cadmium sensitivity. Acta biochimica et biophysica Sinica 44, 886-893.
25. Xu, X. Y., McGrath, S. P., Meharg, A. A., and Zhao, F. J. (2008). Growing rice aerobically markedly decreases arsenic accumulation. Environmental science & technology 42, 5574-5579.
26. Yamaji, N., Xia, J., Mitani-Ueno, N., Yokosho, K., and Feng Ma, J. (2013). Preferential delivery of zinc to developing tissues in rice is mediated by P-type heavy metal ATPase OsHMA2. Plant Physiol 162, 927-939.

1. A genetically modified rice plant or plant cell, comprising a heterologous P_{1B}-type heavy metal ATPase gene operably linked to an OsActin1 promoter; a heterologous ATP-binding cassette (ABC) transporter gene operably linked to an OsActin1 promoter, and a heterologous phytochelatin synthase gene operably linked to an OsActin promoter, wherein the OsActin1 promoter has low activity in a seed endosperm of the modified rice plant compared to its activity in other vegetative tissues of the modified rice plant;
wherein a rice grain of said genetically modified rice plant has reduced arsenic (As) and cadmium (Cd) compared to a control rice plant that has not undergone said genetic modification.

2. The genetically modified rice plant or plant cell according to claim 1, wherein the OsActin1 promoter comprises the nucleic acid sequence set forth in SEQ ID NO: 21 or 31 or a functional sequence variant thereof.

3. The genetically modified rice plant or plant cell according to claim 1, wherein the heterologous P_{1B}-type heavy metal ATPase gene encodes the amino acid sequence set forth in SEQ ID NO: 39; the heterologous ABC transporter gene encodes the amino acid sequence set forth in SEQ ID NO: 37, and the phytochelatin synthase gene encodes the amino acid sequence set forth in SEQ ID NO: 38.

4. The genetically modified rice plant or plant cell according to claim 3, wherein the heterologous P_{1B}-type heavy metal ATPase gene comprises a nucleic acid sequence having at least 80% sequence identity, at least 85% sequence identity, at least 90% sequence identity, at least 95% sequence identity or 100% sequence identity, due to the degeneracy of the genetic code, to the polynucleotide sequence set forth in SEQ ID NO: 22, the heterologous ABC transporter gene comprises a nucleic acid sequence having at least 80% sequence identity, at least 85% sequence identity, at least 90% sequence identity, at least 95% sequence identity or 100% sequence identity, due to the degeneracy of the genetic code, to the polynucleotide sequence set forth in SEQ ID NO: 36, and the heterologous phytochelatin synthase gene comprises a nucleic acid sequence having at least 80% sequence identity, at least 85% sequence identity, at least 90% sequence identity, at least 95% sequence identity or 100% sequence identity, due to the degeneracy of the genetic code, to the polynucleotide sequence set forth in SEQ ID NO: 32.

5. The genetically modified rice plant or plant cell according to claim 1, wherein the exogenous P_{1B}-typ heavy metal ATPase gene, the exogenous ABC transporter gene and/or the exogenous phytochelatin synthase gene are from a cereal crop.

6. The genetically modified rice plant or plant cell according to claim 4, wherein the heterologous P_{1B}-typ heavy metal ATPase gene is OsHMA3 and comprises the nucleic acid sequence set forth in SEQ ID NO: 22, the heterologous ABC transporter gene is OsABCC1 and comprises the nucleic acid sequence set forth in SEQ ID NO: 36, and the heterologous phytochelatin synthase gene is OsPCS1 and comprises the nucleic acid sequence set forth in SEQ ID NO: 32.

7. The genetically modified rice plant or plant cell of claim 6, comprising a heterologous OsHMA3 gene operably linked to an OsActin1 promoter, a heterologous OsABCC1 gene operably linked to an OsActin1 promoter, and a heterologous OsPCS1 gene operably linked to an OsActin1 promoter.

8. The genetically modified rice plant or plant cell of claim 1, of the species Oryza sativa L.

9. A method of creating a genetically modified rice plant, that has reduced arsenic (As) and cadmium (Cd) in its rice grain compared to a rice grain from a control rice plant, the method comprising the steps of:
a) generating a genetically modified rice plant comprising a heterologous P_{1B}-type heavy metal ATPase gene operably linked to an OsActin/promoter;
b) generating a genetically modified rice plant comprising a heterologous ATP-binding cassette (ABC) transporter gene operably linked to an OsActin/promoter;
c) generating a genetically modified rice plant comprising a heterologous phytochelatin synthase gene operably linked to an OsActin1 promoter;
d) select genetically modified rice plants which, respectively, overexpress said exogenous P_{1B}-type heavy metal ATPase gene, said exogenous ATP-binding cassette (ABC) transporter gene and said exogenous phytochelatin synthase gene;
e) crossing two of the three genetically modified rice plants to generate double homozygote plants with respect to the exogenous genes; and
f) crossing a double homozygote plant from step (e) with a third genetically modified rice plant to generate triple homozygote plants that overexpress the exogenous genes.

10. The method according to claim 9, wherein the OsActin1 promoter comprises the nucleic acid sequence set forth in SEQ ID NO: 21 or 31 or a functional sequence variant thereof.

11. The method according to claim 9, wherein: the heterologous P_{1B}-type heavy metal ATPase gene encodes the amino acid sequence set forth in SEQ ID NO: 39, the heterologous ABC transporter gene encodes the amino acid sequence set forth in SEQ ID NO: 37, and the phytochelatin synthase gene encodes the amino acid sequence set forth in SEQ ID NO: 38.

12. A kit for creating a genetically modified rice plant, having reduced arsenic (As) and cadmium (Cd) in its rice grain compared to a rice grain from a control rice plant, wherein the kit comprises bacteria containing vectors comprising a heterologous heavy metal ATPase gene operably linked to an OsActin1 promoter and/or bacteria containing vectors comprising a heterologous ATP-binding cassette (ABC) transporter gene operably linked to an OsActin1 promoter and/or bacteria containing vectors comprising a heterologous phytochelatin synthase gene operably linked to an OsActin1 promoter.

13. The kit of claim 12, wherein the OsActin1 promoter comprises the nucleic acid sequence set forth in SEQ ID NO: 21 or 31 or a functional sequence variant thereof.

14. The kit according to claim 12, wherein the heterologous P_{1B}-type heavy metal ATPase gene encodes the amino acid sequence set forth in SEQ ID NO: 39; the heterologous ABC transporter gene encodes the amino acid sequence set forth in SEQ ID NO: 37, and the phytochelatin synthase gene encodes the amino acid sequence set forth in SEQ ID NO: 38.

15. The kit according to claim 14, wherein the heterologous P_{1B}-type heavy metal ATPase gene comprises a nucleic acid sequence having at least 80% sequence identity, at least 85% sequence identity, at least 90% sequence identity, at least 95% sequence identity or 100% sequence identity, due to the degeneracy of the genetic code, to the polynucleotide sequence set forth in SEQ ID NO: 22, the heterologous ABC transporter gene comprises a nucleic acid sequence having at least 80% sequence identity, at least 85% sequence identity, at least 90% sequence identity, at least 95% sequence identity or 100% sequence identity, due to the degeneracy of the genetic code, to the polynucleotide sequence set forth in SEQ ID NO: 36, and the heterologous phytochelatin synthase gene comprises a nucleic acid sequence having at least 80% sequence identity, at least 85% sequence identity, at least 90% sequence identity, at least 95% sequence identity or 100% sequence identity, due to the degeneracy of the genetic code, to the polynucleotide sequence set forth in SEQ ID NO: 32.

16. The kit according to claim 12, wherein the exogenous P_{1B}-typ heavy metal ATPase gene, the exogenous ABC transporter gene and/or the exogenous phytochelatin synthase gene are from a cereal crop.

17. The kit according to claim 15, wherein the heterologous P_{1B}-typ heavy metal ATPase gene is OsHMA3 and comprises the nucleic acid sequence set forth in SEQ ID NO: 22, the heterologous ABC transporter gene is OsABCC1 and comprises the nucleic acid sequence set forth in SEQ ID NO: 36, and the heterologous phytochelatin synthase gene is OsPCS1 and comprises the nucleic acid sequence set forth in SEQ ID NO: 32.
